SEQUENCE LISTING (25 sequences)

SEQ ID NO: 1 - RNA, 21 nt, Artificial Sequence
  hsa-miR-27a-3p
uucacaguggcuaaguuccgc

SEQ ID NO: 2 - RNA, 21 nt, Artificial Sequence
  hsa-miR-27b-3p
uucacaguggcuaaguucugc

SEQ ID NO: 3 - RNA, 22 nt, Artificial Sequence
  hsa-miR-29c-3p
uagcaccauuugaaaucgguua

SEQ ID NO: 4 - RNA, 23 nt, Artificial Sequence
  hsa-miR-30c-5p
uguaaacauccuacacucucagc

SEQ ID NO: 5 - RNA, 22 nt, Artificial Sequence
  hsa-miR-122-5p
uggagugugacaaugguguuug

SEQ ID NO: 6 - RNA, 22 nt, Artificial Sequence
  hsa-miR-132-3p
uaacagucuacagccauggucg

SEQ ID NO: 7 - RNA, 23 nt, Artificial Sequence
  hsa-miR-142-3p
uguaguguuuccuacuuuaugga

SEQ ID NO: 8 - RNA, 21 nt, Artificial Sequence
  hsa-miR-143-3p
ugagaugaagcacuguagcuc

SEQ ID NO: 9 - RNA, 22 nt, Artificial Sequence
  hsa-miR-148a-3p
ucagugcacuacagaacuuugu

SEQ ID NO: 10 - RNA, 22 nt, Artificial Sequence
  hsa-miR-193b-3p
aacuggcccacaaagucccgcu

SEQ ID NO: 11 - RNA, 21 nt, Artificial Sequence
  hsa-miR-195-5p
uagcagcacagaaauauuggc

SEQ ID NO: 12 - RNA, 23 nt, Artificial Sequence
  hsa-miR-199a-3p
cccaguguucagacuaccuguuc

SEQ ID NO: 13 - RNA, 22 nt, Artificial Sequence
  hsa-miR-320a-3p
aaaagcuggguugagagggcga

SEQ ID NO: 14 - RNA, 22 nt, Artificial Sequence
  hsa-miR-324-5p
cgcauccccuagggcauuggug

SEQ ID NO: 15 - RNA, 23 nt, Artificial Sequence
  hsa-miR-335-5p
ucaagagcaauaacgaaaaaugu

SEQ ID NO: 16 - RNA, 22 nt, Artificial Sequence
  hsa-miR-340-5p
uuauaaagcaaugagacugauu

SEQ ID NO: 17 - RNA, 22 nt, Artificial Sequence
  hsa-miR-365a-3p
uaaugccccuaaaaauccuuau

SEQ ID NO: 18 - RNA, 21 nt, Artificial Sequence
  hsa-miR-378a-3p
acuggacuuggagucagaagg

SEQ ID NO: 19 - RNA, 22 nt, Artificial Sequence
  hsa-miR-483-5p
aagacgggaggaaagaagggag

SEQ ID NO: 20 - RNA, 21 nt, Artificial Sequence
  hsa-miR-486-3p
cggggcagcucaguacaggau

SEQ ID NO: 21 - RNA, 22 nt, Artificial Sequence
  hsa-miR-486-5p
uccuguacugagcugccccgag

SEQ ID NO: 22 - RNA, 22 nt, Artificial Sequence
  hsa-miR-532-5p
caugccuugaguguaggaccgu

SEQ ID NO: 23 - RNA, 21 nt, Artificial Sequence
  hsa- miR-652-3p
aauggcgccacuaggguugug

SEQ ID NO: 24 - RNA, 22 nt, Artificial Sequence
  hsa-miR-885-5p
uccauuacacuacccugccucu

SEQ ID NO: 25 - RNA, 22 nt, Artificial Sequence
  hsa- let-7e-5p
ugagguaggagguuguauaguu

(19) United States
(12) Patent Application Publication
(10) Pub. No.: US 2024/0093297 A1
(43) Pub. Date: Mar. 21, 2024

(54) Biomarkers For Cognitive Conditions

(71) Applicant: OTAGO INNOVATION LIMITED, Dunedin (NZ)

(72) Inventors: Diane Guévremont, Dunedin (NZ); Joanna Margaret Williams, Dunedin (NZ); Warren Perry Tate, Dunedin (NZ); Wickliffe Carson Abraham, Dunedin (NZ)

(21) Appl. No.: 18/006,566

(22) PCT Filed: Jul. 23, 2021
(86) PCT No.: PCT/NZ2021/050113

§ 371 Date: Jan. 23, 2023

(30) Foreign Application Priority Data
Jul. 24, 2020 (NZ) 766493

(51) Int. Cl. C12Q 1/6883; C12Q 1/6851

(52) U.S. Cl. CPC C12Q 1/6883; C12Q 1/6851; C12Q 2600/112; C12Q 2600/158; C12Q 2600/178

(57) ABSTRACT

Analysis of microRNA (miRNA) signatures to determine or predict stages of cognitive impairment and likelihood of Alzheimer's disease in an individual.

### FIELD OF THE INVENTION

The present invention relates to a microRNA (miRNA) signature that can be used to determine or predict the stage of cognitive impairment and likelihood of Alzheimer's disease in an individual. This information can be paired with preventative and active therapies to prevent or delay cognitive decline.

### BACKGROUND TO THE INVENTION

Early detection of Alzheimer's disease (AD) is critical to the development of and delivery of effective treatment strategies. While it is feasible to identify known mutations in family pedigrees with a history of AD, no such routine test is available to detect the sporadic form of the disease. ApoEε4 genotype is a long-established risk factor for late-onset AD, but by itself it is not strongly predictive of progression to AD and no other single mutation has demonstrated a stronger predictive value. By contrast, while it is possible to quantify levels of amyloid-β (Aβ), one of the diagnostic features of AD, in cerebrospinal fluid and brain, as well as anatomical changes in cortical structures, it is not possible to measure these early changes without repeated use of positron emission tomography (PET) scans, magnetic resonance imaging (MRI) scans or lumbar puncture. These are expensive and/or invasive procedures that are only available in highly specialized centres and not currently suitable for population screening.

Attention has focused on blood-borne biomarkers of AD, yet no such biomarker is currently available for predicting disease onset. Indeed, despite the strong association between amyloidosis and cognitive decline, there is much debate as to whether their levels in blood plasma correlate well with the disease. Direct measurement of Aβ and phospho-tau has been immensely technically challenging, despite development of new technologies. This is likely due to the highly aggregative nature of Aβ, the low levels in blood, and a lack of knowledge as to how Aβ is exported from the brain. Moreover, there is evidence that Aβ levels in the blood decline with AD progression.

Recently, a growing body of evidence suggests that microRNA (miRNA), a class of non-coding RNA that function by regulating gene expression at the post-transcriptional level, are dysregulated in AD and that blood-borne miRNA may be good candidate biomarkers of the disease. Interestingly, while miRNA can be detected in cerebrospinal fluid, miRNA cross the blood brain barrier and are protected from degradation by association with protein complexes and sequestration into membrane bound vesicles, such as exosomes.

Indeed, recent evidence suggest that exosomes may be involved in propagation of neurodegenerative disease and that exosome-derived miRNA can transduce recipient cells. Therefore, circulating levels of miRNA may not only accurately reflect neuronal function and dysfunction, but may represent novel therapeutic targets for the treatment of dementia.

There is little consistency in the miRNA species that are reported to make up putative AD-associated panels. Alongside heterogeneity within study cohorts, preanalytical variation in the collection, processing and storage of blood, variation in blood fractions and the analytical and statistical platforms used to assess biomarker levels are crucial limiting factors in the search for blood-based biomarkers of AD.

It is therefore vital to identify robust, easily monitored biomarkers that are accurate indicators of incipient AD and understand how these change over the entire course of the disease. It is an object of the present disclosure to go some way to meeting this need; and/or to at least provide the public with a useful choice.

Other objects of the invention may become apparent from the following description which is given by way of example only.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

The invention provides methods of detecting an elevated cognitive impairment biomarker panel comprising: a) detecting the levels of any of the miRNA biomarkers listed in Table 1 (and refer FIG. 1) in any combination in a body fluid sample from a human; and b) detecting said elevated cognitive impairment biomarker panel when the level of at least one of said miRNA biomarkers is upregulated or downregulated relative to a healthy control level. In some embodiments, the miRNA biomarkers include miR-29c-3p, miR-335-5p, miR-142-3p, miR-324-5p, miR-195-5p, miR-148a-3p, miR-27a-3p, miR-27b-3p, miR-122-5p, miR-193b-3p, miR-342-3p and miR-885-5p. In some embodiments, the miRNA biomarkers further include miR-143-3p, miR-320a-3p, miR-365-3p, miR-532-5p, and miR-132-3p.

In some embodiments, step a) comprises detecting the levels of any of the miRNA biomarkers listed in Table 1 in any combination in the body fluid. In some embodiments, the miRNA biomarkers include miR-29c, miR-335-5p, miR-142-3p, miR-324-5p, miR-195-5p, miR-148-3p, miR-27a-3p, miR-27b-3p, miR-122-5p, miR-193b-3p, miR-342-3p and miR-885-5p.

In some embodiments, the human is suspected of having cognitive impairment or Alzheimer's Disease, e.g., as determined by cognitive testing. In some embodiments, the body fluid sample is plasma. In some embodiments, the body fluid is selected from serum, white blood cells, or whole blood.

In some embodiments, detecting the levels of miRNA biomarkers comprises detecting by an amplification-based method. In some embodiments, detecting the levels of miRNA biomarkers comprises detecting by an array-based method.

In some embodiments, the human is diagnosed as likely having a high amyloid-p load in the brain, amyloid positive (Aβ+) when any one of miR-29c-3p, miR-335-5p, or miR-142-3p is upregulated, or miR-122-5p, miR-342-3p, miR-885-5p is downregulated. In some embodiments, the method further comprises detecting the levels of miRNA biomarkers miR-27b-3p, miR-143-3p, miR-320a-3p, miR-532,5p, miR-193-3p, miR-324-5p, miR-365-3p, miR-148-3p, miR-27a-3p, or miR-132-3p. In some embodiments, amyloid-Aβ load is correlated with levels of expression of miR-27a-3p, miR-27b-3p, and miR-324-5p.

In some embodiments, the human is diagnosed with mild cognitive impairment (MCI) when any one of miR-195-5p, miR-148-3p, miR-324-5p is upregulated or miR-142-3p is downregulated. In some embodiments, the method further comprises detecting the levels of miRNA biomarkers miR-885-5p, miR-483-5p, miR-199a-3p, mir-365-3p, miR-132-3p, miR-27a-3p, miR-27b-3p, miR-143-3p, miR-335-5p, or let-7e-5p.

In some embodiments, the human is diagnosed with Alzheimer's Disease when any one of miR-122-5p, miR-193b-3p, or miR-885-5p is upregulated or any one of miR-27a-3p, miR-27b-3p, or miR-324-5p is downregulated. In some embodiments, the method further comprises detecting the levels of miRNA biomarkers miR-486-3p, miR-486-5p, miR-378-3p, miR-365-3p, miR-132-3p, miR-195-5p, miR-335-5p, miR-30c-5p, miR-340-5p, or miR-142-3p.

In some embodiments, the method further comprises administering a PET or MRI scan, or cognitive therapy to the human when an elevated cognitive impairment biomarker panel is detected. In some embodiments, drug therapy is also administered to the human when an elevated cognitive impairment biomarker panel is detected.

In some embodiments, the method further comprises obtaining cerebral spinal fluid via a lumbar puncture (a spinal tap sample) from the human and detecting the level of amyloid-β or tau/p-tau in the sample when a cognitive impairment biomarker panel is detected. In some embodiments, the method comprises obtaining serum, white blood cells, or whole blood from the human and detecting the level of amyloid-β or tau/p-tau in the sample when a cognitive impairment biomarker panel is detected. In some embodiments, the method further comprises detecting the presence of the ApoE-εE4 genotype in a body fluid or tissue sample.

In some embodiments the method further comprises detecting the level of amyloid-β or tau/p-tau in a sample taken from a human when a cognitive impairment biomarker panel is detected.

The invention also provides methods of measuring an elevated cognitive impairment biomarker panel in a human comprising: a) obtaining a body fluid sample from the human; b) determining a measurement for the panel of biomarkers in the biological sample, selected from the miRNA biomarkers listed in Table 1 in any combination, wherein the measurement comprises measuring a level of each biomarker in the panel. In some embodiments, the panel comprises miR-29c-3p, miR-335-5p, miR-142-3p, miR-324-5p, miR-195-5p, miR-148-3p, miR-27a-3p, miR-27b-3p, miR-122-5p, miR-193b-3p, miR-342-3p and miR-885-5p. In some embodiments, the miRNA biomarkers further include miR-143-3p, miR-320-3p, miR-365-3p, miR-532-5p, and miR-132-3p.

In some embodiments, the human is suspected of having cognitive impairment or Alzheimer's Disease. In some embodiments, the body fluid sample is plasma. In some embodiments, the body fluid is selected from serum, white blood cells, or whole blood.

In some embodiments, the determining comprises measuring by an amplification-based method. In some embodiments, the determining comprises measuring by an array-based method.

In some embodiments, the human is diagnosed as likely having a high amyloid-β load in the brain, amyloid positive (Aβ+) when any one of miR-29c-3p, miR-335-5p, or miR-142-3p is upregulated, or miR-122-5p, miR-342-3p, miR-885-5p is downregulated relative to a healthy control. In some embodiments, the method further comprises detecting the levels of miRNA biomarkers miR-27b-3p, miR-143-3p, miR-320a-3p, miR-532,5p, miR-193-3p, miR-324-5p, miR-365-3p, miR-148-3p, miR-27a-3p, or miR-132-3p. In some embodiments, amyloid-β load is correlated with levels of expression of miR-27a-3p, miR-27b-3p, and miR-324-5p.

In some embodiments, the human is diagnosed with mild cognitive impairment (MCI) when any one of miR-195-5p, miR-148-3p, miR-324-5p is upregulated or miR-142-3p is downregulated relative to a healthy control. In some embodiments, the method further comprises detecting the levels of miRNA biomarkers miR-885-5p, miR-483-5p, miR-132-3p, miR-199a-3p, mir-365-3p, miR-132-3p, miR-27a-3p, miR-27b-3p, miR-143-3p, miR-335-5p, or let-7e-5p.

In some embodiments, the human is diagnosed with Alzheimer's Disease when any one of miR-122-5p, miR-193b-3p, or miR-885-5p is upregulated or any one of miR-27a-3p, miR-27b-3p, or miR-324-5p is downregulated relative to a healthy control. In some embodiments, the method further comprises detecting the levels of miRNA biomarkers miR-486-3p, miR-486-5p, miR-378-3p, miR-365-3p, miR-132-3p, miR-195-5p, miR-335-5p, miR-30c-5p, miR-340-5p, or miR-142-3p.

In some embodiments, the method further comprises administering a PET or MRI scan, or cognitive therapy to the human when an elevated cognitive impairment biomarker panel is detected. In some embodiments, drug therapy is also administered to the human when an elevated cognitive impairment biomarker panel is detected.

In some embodiments, the method further comprises obtaining a spinal tap sample from the human and detecting the level of amyloid or tau in the sample when an elevated cognitive impairment biomarker panel is detected. In some embodiments, the method further comprises detecting the presence of the ApoE-c4 genotype in the body fluid sample.

The invention also provides methods of determining progression of cognitive impairment comprising
a) obtaining a first body fluid sample from a human at a first time;
b) obtaining a second body fluid sample from the human at a second time that is after the first time;
c) detecting the levels of miRNA biomarkers miR-29c-3p, miR-335-5p, miR-142-3p, miR-324-5p, miR-195-5p, miR-148a-3p, miR-27a-3p, miR-27b-3p, miR-122-5p, miR-193b-3p, miR-342-3p and miR-885-5p in the first body fluid sample;
d) detecting the levels of miRNA biomarkers miR-29c-3p, miR-335-5p, miR-142-3p, miR-324-5p, miR-195-5p, miR-148-3p, miR-27a-3p, miR-27b-3p, miR-122-5p, miR-193b-3p, miR-342-3p and miR-885-5p in the second body fluid sample;
e) comparing the levels of the miRNA biomarkers taken at the first time, thereby determining progression of cognitive impairment.

In one embodiment, the human is diagnosed as likely having a high amyloid-β load in the brain when miR-29c-3p and miR-335-5p are altered.

In one embodiment, the human is diagnosed with mild cognitive impairment (MCI) when miR-142-3p, miR-324-5p, miR-195, miR-148a-3p are altered.

In one embodiment, the human is diagnosed with Alzheimer's Disease when miR-27a-3p, miR-27b-3p, miR-122-5p, miR-193b-3p, miR-324-5p are altered.

The invention also provides kits for detecting a cognitive impairment biomarker panel. In some embodiments, the kit comprises: a) oligonucleotides that specifically hybridize to each of miRNA biomarkers miR-29c-3p, miR-335-5p, miR-142-3p, miR-324-5p, miR-195-5p, miR-148-3p, miR-27a-3p, miR-27b-3p, miR-122-5p, miR-193b-3p, miR-342-3p and miR-885-5p; and b) labelled probes that specifically detect each of miRNA biomarkers miR-29c-3p, miR-335-5p, miR-142-3p, miR-324-5p, miR-195-5p, miR-148-3p, miR-27a-3p, miR-27b-3p, miR-122-5p, miR-193b-3p, miR-342-3p and miR-885-5p. In some embodiments, the kit further comprises oligonucleotides that specifically hybridize to miR-143-3p, miR-320-3p, miR-365-3p, miR-532-5p, and miR-132-3p and labelled probes that specifically detect miR-143-3p, miR-320-3p, miR-365-3p, miR-532-5p, and miR-132-3p. In some embodiments, the oligonucleotides or probes are attached to an array.

In some embodiments, the kit includes separate reaction mixtures or separate arrays for detecting the cognitive impairment biomarkers for MCI and AD. In some embodiments, the kit further includes reagents for detecting or measuring the levels of the presently described cognitive impairment biomarkers, e.g., buffers, polymerase, etc.

In some embodiments, the kit further includes reagents for detecting the presence of an ApoE-249 4 allele, or amyloid-β, or tau/p-tau levels.

The invention also provides methods of determining the likelihood that a human likely has a high amyloid-β load in the brain, amyloid positive (Aβ+), comprising detecting the levels of any of the miRNA biomarkers listed in Table 1 in any combination (e.g., miR-29c-3p, miR-335-5p, miR-142-3p, miR-324-5p, miR-195-5p, miR-148-3p, miR-27a-3p, miR-27b-3p, miR-122-5p, miR-193b-3p, miR-342-3p and miR-885-5p) in a body fluid sample from the human and determining that the human likely has a high amyloid-β load in the brain, amyloid positive (Aβ+), when any of miR-29c-3p, miR-335-5p, miR-142-3p are upregulated or miR-122-5p, miR-342-3p and miR-885-5p are downregulated relative to a healthy control level.

The invention also provides methods of determining the likelihood that a human has mild cognitive impairment (MCI) comprising detecting the levels of any of the miRNA biomarkers listed in Table 1 in any combination (e.g., miR-29c-3p, miR-335-5p, miR-142-3p, miR-324-5p, miR-195-5p, miR-148-3p, miR-27a-3p, miR-27b-3p, miR-122-5p, miR-193b-3p, and miR-885-5p) in a body fluid sample from the human and determining that the human likely has MCI when any one of miR-195-5p, miR-148-3p, miR-324-5p are upregulated or miR-142-3p is downregulated relative to a healthy control level.

In invention also provides methods of determining the likelihood that a human has Alzheimer's Disease (AD) comprising detecting the levels of any of the miRNA biomarkers listed in Table 1 in any combination (e.g., miR-29c-3p, miR-335-5p, miR-142-3p, miR-324-5p, miR-195-5p, miR-148-3p, miR-27a-3p, miR-27b-3p, miR-122-5p, miR-193b-3p, and miR-885-5p) in a body fluid sample from the human and determining that the human likely has AD when any one of miR-122-5p, miR-193b-3p, or miR-885-5p is upregulated or any one of miR-27a-3p, miR-27b-3p, or miR-324-5p is downregulated relative to a healthy control level.

TABLE 1
Differentially expressed miRNA. Significantly differentially expressed
miRNA were identified in each cohort using empirical Bayes moderated
t-tests (p < 0.05), based on fold changes relative to HC (healthy
controls). In bold are the statistically significant miRNA expression
in a particular cohort; p < 0.05. Aβ+, cognitively normal
amyloid positive; MCI, mild cognitive impairment; AD, Alzheimer's
disease. Number of participants in each cohort are in parentheses.

| | AIBL | AIBL | Pmed | Otago | AIBL |
|---|---|---|---|---|---|
| target | Aβ+ (21) | MCI (38) | MCI (36) | AD (44) | AD (21) |
| miR-122-5p | −1.48 | −1.08 | 1.68 | 2.48 | 2.09 |
| miR-885-5p | −1.50 | 1.95 | 2 | 2.1 | 1.9 |
| miR-486-3p | −1.44 | −1.27 | 1.47 | 2.18 | 1.12 |
| miR-193b-3p | −1.32 | −1.1 | 1.02 | 1.58 | 1.6 |
| miR-378-3p | −1.40 | −1.06 | 1.91 | 1.71 | 1.15 |
| miR-486-5p | −1.59 | −1.03 | 2.53 | 1.49 | 1.1 |
| miR-320a-3p | −1.22 | −1.09 | 1.55 | 1.32 | 1.21 |
| miR-425-5p | −1.36 | −1.17 | 1.6 | 1.29 | 1.02 |
| miR-342-3p | −1.34 | −1.1 | 1.26 | 1.24 | 1.11 |
| miR-532-5p | −1.21 | −1.01 | 1.35 | 1.23 | 1.2 |
| miR-365-3p | −1.13 | 1.11 | 1.86 | 1.24 | 1.41 |
| miR-132-3p | 1.06 | 1.19 | 1.45 | 1.24 | 1.32 |
| miR-195-5p | 1.22 | 1.34 | 1.52 | 1.19 | 1.15 |
| miR-335-5p | 1.89 | 1.85 | 1.53 | 1.08 | 1.28 |
| miR-148a-3p | 1.29 | 1.45 | 1.41 | 1.08 | 1.07 |
| miR-199a-3p | 2.15 | 1.79 | 1.14 | 1.01 | 1.36 |
| miR-340-5p | 1.55 | 1.28 | −1.02 | 1.29 | 1.06 |
| miR-30c-5p | 2.04 | 1.32 | −1.89 | 1.12 | 1.52 |
| miR-29c-3p | 1.51 | 1.59 | −1.16 | 1.02 | 1.33 |
| miR-143-3p | 1.45 | 1.29 | 1.08 | −1.04 | 1.14 |
| miR-20b-5p | −1.41 | 1.05 | 1.14 | 1.54 | −1.1 |
| miR-93-3p | −1.26 | −1.08 | 1.17 | 1.42 | −1.12 |
| miR-92a-3p | −1.31 | 1 | 1.63 | 1.11 | −1.07 |
| miR-93-5p | −1.23 | −1.11 | 1.1 | 1.1 | −1.13 |
| let-7e-5p | 1.46 | −1.25 | −2.34 | 1.08 | −1.06 |
| miR-186-5p | −1.21 | −1.22 | 1.19 | 1.1 | −1.14 |
| miR-483-5p | −1.44 | 1.85 | 1.92 | 1.06 | −1.11 |
| miR-142-3p | 2.34 | −1.49 | −2.02 | −1.04 | −1.21 |
| miR-652-3p | 1.29 | 1.2 | 1.07 | −1.07 | −1.01 |
| miR-324-5p | 1.25 | −1.18 | −1.25 | −1.24 | −1.31 |
| miR-27b-3p | 1.38 | 1.29 | 1.32 | −1.27 | −1.26 |
| miR-27a-3p | 1.13 | 1.11 | 1.24 | −1.29 | −1.28 |

In some embodiments, the body fluid is plasma. In some embodiments, further comprising detecting the presence of the ApoE-ε4 genotype in the body fluid sample.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### BRIEF DESCRIPTION OF THE FIGURES

The present invention will be described with reference to the accompanying figures.

FIG. 1—Sequence listing

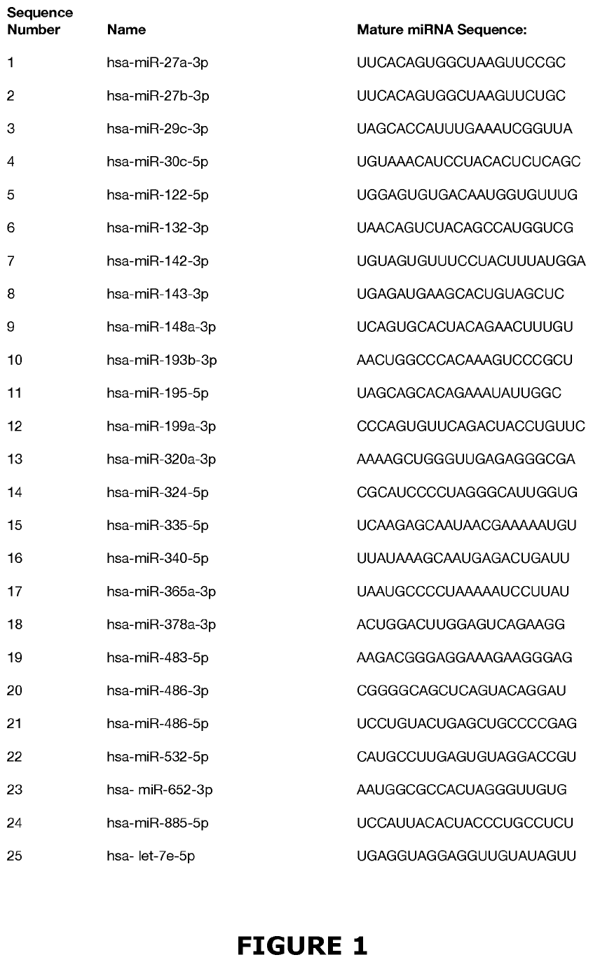

FIG. 2—Flowchart of the study plan.

FIG. 3—Forest Plots of the weighted fold change in miRNA expression for Aβ+, MCI, and AD cross-sectional cohorts: The linear mixed effects model included Aβ+ (n=21) and pooled results for the MCI (n=74) and AD (n=63) cohorts. Observed outcomes for each disease stage are represented with a diamond (Aβ+=gold, MCI=orange, AD=crimson). The width of the diamond reflects the precision of the estimate (95% CI); the weights correspond to the inverse standard deviations of the effect size estimates from the studies; the position on the x-axis represents the measure estimate (Fold Change), with the horizontal line indicating “no change” in microRNA expression. A positive effect size represents upregulation and a negative effect size represents downregulation in microRNA expression. Data are relative to HC groups. Summary estimates are provided in Table 5.

FIG. 4—Venn Diagram: Showing the association of the 16 miRNAs retained after meta-analyses with disease stage.

FIG. 5—Consensus ranking of miRNAs and diagnostic value of miRNA: a) Each of the 16 miRNA identified in the meta-analysis were ranked using 3 independent criteria (see Table 6). (b) The diagnostic ability of each signature (in bold) was assessed by computing the AUC value of the ROC curve (logistic regression with normalised Ct values, compared to the HC group. The results of each ROC analyses are shown in (c).

FIG. 6—Box and whisker plots of biomarker miRNA expression in AIBL longitudinal cohort: Expression of biomarker miRNA was studied in the AIBL longitudinal cohort (n=21; Aβ+ to MCI stage and n=18 MCI to AD stage; total MCI=39). The lines within the boxes show the median miRNA expression (normalised Ct values) and the whiskers represent the 95% CI. Significant differences were identified using generalized estimating equations (*p<0.05; ** p<0.01; *** p<0.001). The hashed line represents the median values in the AIBL HC group and were not included in the longitudinal analysis.

FIG. 7—Bioinformatics: show pathways targeted by (a) Aβ+, MCI and AD-related microRNA (refer Table 6), (b) the combined list of biomarker microRNA and (c) those correlated with centiloid values (amyloidosis).

### DETAILED DESCRIPTION OF THE INVENTION

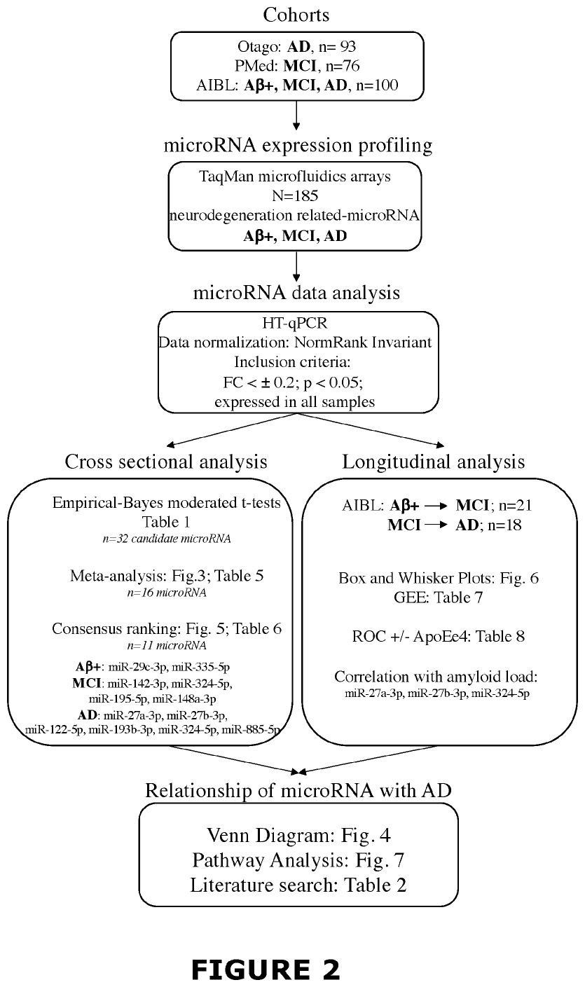

We have shown that specific plasma miRNA are dynamically altered with amyloidosis in the APP_{swe}/PSEN1dE9 transgenic mouse model of AD. Building on this and other studies reinforcing the complex origin of AD, we sought to collect a standardized biofluid (plasma) and use a robust miRNA analysis platform (quantitative PCR TaqMan microfluidics arrays) to identify distinctive miRNA-based biomarkers effectively reflecting the various phases of cognitive disease progression (refer FIG. 2).

Accordingly, we assessed the levels of miRNA in blood plasma of a well characterised cohort of people with AD, mild cognitive impairment (MCI), cognitively normal yet Aβ positive (Aβ+), and age and sex-matched elderly people, using TaqMan microfluidics arrays. We identified a group of miRNA which are consistently altered with AD or MCI regardless of preanalytical processing, but importantly have shown that the levels of particular microRNA are dynamic throughout the progression of the disease. The presently described results show that it is possible to identify disease-associated miRNA altered in blood plasma, and that the miRNA signature changes over the course of AD progression.

### Definitions

The term “cognitive impairment biomarker” refers to a biomarker that can be used to assess the likelihood that an individual has or will develop significant amyloid levels, cognitive impairment, or AD. A biomarker can be presence of, absence of, or differential expression of a specific miRNA, mRNA, or protein. A biomarker can also be a modified version of miRNA, RNA (splice variant), DNA (e.g., methylated), or protein (e.g., phosphorylated), or represent a mutated or allelic variant of miRNA, RNA, DNA, or protein. The cognitive impairment biomarker panels described herein can include the miRNA biomarkers shown in Table 1 in any combination, and optionally ApoE-ε4 and amyloid beta.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.” A nucleic acid monomer “nucleotide” refers to a nucleoside further comprising a “backbone moiety”. A backbone moiety covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

The phrase “selectively (or specifically) hybridizes to” refers to the binding, duplexing, or hybridizing of a molecule predominantly (e.g., at least 50% of the hybridizing molecule) to a particular nucleotide sequence under stringent hybridization conditions when that sequence is present in a complex mixture (e.g., total cellular or library DNA or RNA). Polynucleotide primers specifically hybridize to a polynucleotide template in an amplification reaction (e.g., at an annealing temperature of about 60 C) when the primers amplify the template in a reaction mixture comprising a complex mixture of polynucleotides (e.g., isolated from a cell) to produce an amplification product that is at least the most predominant amplification product and is preferably the only significant (e.g., representing at least 90-95% of all amplification products in the sample) amplification product of the reaction (see, e.g., Sambrook et al., Molecular Cloning: A Laboratory Manual (Cold Spring Harbor Laboratory Press, New York, N.Y., 2nd ed. 1989))

The terms “identical” or “100% identity,” in the context of two or more nucleic acids or polypeptide sequences, refer to two or more sequences or subsequences that are the same sequences. Two sequences are “substantially identical” or a certain percent identity if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. Typically, one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Examples of an algorithm that is suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information at the website ncbi.nlm.nih.gov.

The terms “vessel,” “tube,” “container,” “microwell,” etc refer to objects that hold a reaction or reagents, e.g., in a kit.

The term “prognosis,” “prognostication,” “prognostic,” “prediction,” “predict,” “predictive,” “diagnosis,” “diagnostic,” and related terms are used herein in reference to individuals to denote processes and results of estimating outcomes of cognitive function, including the probability of progression, e.g. to AD. These terms are also included in the scope of the terms “assess,” “assessment,” “assessing” and the related terms. It is to be understood that various measures of prognosis and outcome prediction can be used, such as probability of cognitive decline, and that a prognosis and/or predictions are often expressed as estimates or probabilities, and are not always precise.

A “control” sample or value refers to a sample that serves as a reference, usually a known reference, for comparison to a test sample or test conditions. For example, a test sample can be taken from a test condition, e.g., from an individual showing signs of cognitive decline and compared to samples from known conditions, e.g., from a healthy or cognitively normal individual (negative control), or from an individual known to have MCI or AD (positive control). A control can also represent an average value or a range gathered from a number of tests or results. A control can also be prepared for reaction conditions. For example, a positive control for the presence of nucleic acid could include primers or probes that will detect a sequence known to be present in the sample, while a negative control would be free of nucleic acids. One of skill in the art will recognize that controls can be designed for assessment of any number of parameters. Controls can be designed for in vitro applications. One of skill in the art will understand which controls are valuable in a given situation and be able to analyse data based on comparisons to control values. Controls are also valuable for determining the significance of data. For example, if values for a given parameter are widely variant in controls, variation in test samples will not be considered as significant.

The term “therapy” is used herein synonymously with the term “treatment.” A therapy may include one or more types of therapy. For example, a therapy may include a combination of cognitive therapy and drug therapy. A therapy or treatment can be administered one or more times over a certain period of time, followed by a period during which no treatment or therapy is administered. A therapy cycle can last for days or weeks (in one example, four weeks). One or more cycles of therapy or treatment can be administered. For example, one, two, three, four, five, six, seven, eight, nine or ten cycles of therapy or treatment can be administered. The therapy may be the same or varied during different cycles, e.g., depending on response. During a therapy cycle, the therapies may be administered on a single day, several consecutive days, or continuously as an outpatient or as an inpatient. A therapy may last minutes, hours, or days, depending on the specific protocol. Therapy cycle may repeat weekly, bi-weekly, or monthly. A therapy cycle can include one or more therapy sessions. One or more therapy cycles can be referred collectively as a “course” of therapy.

The term “comprising” as used in this specification and claims means “consisting at least in part of”. When interpreting each statement in this specification and claims that includes the term “comprising”, features other than that or those prefaced by the term may also be present. Related terms such as “comprise”, “comprised” and “comprises” are to be interpreted in the same manner.

As used herein the term “and/or” means “and” or “or”, or both.

As used herein “(s)” following a noun means the plural and/or singular forms of the noun.

The general chemical and biological terms used, for example, in the formulae herein have their usual meanings.

### miRNA and Detection Thereof

MicroRNAs (miRNAs) are small RNAs of 17-25 nucleotides, which function as regulators of gene expression in eukaryotes. miRNAs are initially expressed in the nucleus as part of long primary transcripts called primary miRNAs (pri-miRNAs). These are processed into mature miRNAs, which are the active molecules that can target the miRNA to the 3′ untranslated region (3′-UTR) of a target mRNA.

A particular miRNA may be referred to as a miRNA molecule, a miR, or an equivalent thereof or a source or a precursor thereof. Some miRNA molecules are encoded by several precursors. It is also possible that one precursor may lead to several mature miRNA molecule. The term “miRNA,” unless otherwise indicated, refers to the processed miRNA, after it has been cleaved from its precursor.

Extracellular miRNAs freely circulate in a wide range of bodily fluids. Accordingly, in some embodiments, the biological sample used for determining the level of one or more miRNA biomarkers is a bodily fluid, such as blood, serum, plasma, urine, saliva, tears, sweat, semen, vaginal secretions, lymph, bronchial secretions, or CSF. In some embodiments, the sample is obtained from a bodily fluid other than CSF, in particular, plasma.

The level of one or more miRNA biomarkers in a biological sample may be determined by any suitable method. Generally, miRNA can be detected and quantified from a sample, such as samples of isolated RNA by various methods known for mRNA detection, including, for example, amplification-based methods (e.g., Polymerase Chain Reaction (PCR), Real-Time Polymerase Chain Reaction (RT-PCR), Quantitative Polymerase Chain Reaction (qPCR), rolling circle amplification, etc.), hybridization-based methods (e.g., hybridization arrays (e.g., microarrays), NanoString analysis, Northern Blot analysis, branched DNA (bDNA) signal amplification, and in situ hybridization), and sequencing-based methods (e.g. next-generation sequencing methods, for example, using the Illumina or IonTorrent platforms). Other exemplary techniques include ribonuclease protection assay (RPA) and mass spectroscopy (see, e.g., Zhang et al. MicroRNA Detection and Pathological Functions, Chpt. 1.4, Springer 2015).

In some embodiments, RNA is converted to DNA (cDNA) prior to analysis. cDNA can be generated by reverse transcription of isolated miRNA using conventional techniques. miRNA reverse transcription kits are known and commercially available. Examples of suitable kits include, but are not limited to the mirVana TaqMan miRNA transcription kit (Ambion, Austin, Texas USA), and the TaqMan miRNA transcription kit (Applied Biosystems, Foster City, Calif.). Universal primers, or specific primers, including miRNA-specific stem-loop primers, are known and commercially available, for example, from Applied Biosystems. In some embodiments, miRNA is amplified prior to measurement. In other embodiments, the level of miRNA is measured during the amplification process. In still other embodiments, the level of miRNA is not amplified prior to measurement. Some exemplary methods suitable for determining the level of miRNA in a sample are described in greater detail below. These methods are provided by way of illustration only, and it will be apparent to a skilled person that other suitable methods may likewise be used. Many amplification-based methods exist for detecting the level of miRNA nucleic acid sequences, including, but not limited to, PCR, RT-PCR, qPCR, and rolling circle amplification. Such methods can also be used to detect DNA or mRNA, e.g., ApoE-ε4. Other amplification-based techniques include, for example, ligase chain reaction, multiplex ligatable probe amplification, in vitro transcription (IVT), strand displacement amplification, transcription-mediated amplification, RNA (Eberwine) amplification, and other methods that are known to persons skilled in the art.

Kits for quantitative real time PCR of miRNA are known, and are commercially available. Examples of suitable kits include, but are not limited to, the TaqMan miRNA Assay (Applied Biosystems) and the mirVana qRT-PCR miRNA detection kit (Ambion). The miRNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence.

The presently described miRNAs can be separated and/or detected using miRNA arrays, which are ordered macroarrays or microarrays of nucleic acid molecules (probes) that are fully or nearly complementary or identical to a plurality of miRNA molecules or precursor miRNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art.

Also included in the present description are kits for detecting a cognitive impairment biomarker panel. The kit can include oligonucleotides that specifically hybridize to any of the biomarkers listed in Table 1 in any combination. In some embodiments, the kit includes labeled probes (e.g. fluorescently, or otherwise non-naturally labeled). In some embodiments, the kit includes reagents for amplification, e.g., RT-PCR, such as buffers and polymerase(s).

The kits described herein can be designed for multiplex detection, with biomarkers associated with amyloid beta, cognitive impairment, and AD in separate vessels. The kits can include at least one microarray, e.g., for detecting the cognitive impairment biomarkers described herein. A kit can also include consumables (e.g. reaction vessels, reagents) and instruction for use.

### Diagnosis and Prediction of Cognitive Impairment and Alzheimer's Disease

The presently described biomarker panel seeks to provide a more quantitative method of predicting the progression of a cognitive disorder and AD in an individual. Currently, dementia and AD are detected by noticing confusion, forgetfulness, social withdrawal, loss of visual or spatial understanding, or mood changes in an individual.

Several cognitive tests are also available, including the Mini-Mental State Examination (MMSE), Mini-Cog test, Addenbrooke's Cognitive Examination—Revised (ACE-R), and Montreal Cognitive Assessment. Such tests are advantageously employed in combination with the presently described biomarker panel.

Cognitive therapy has been shown to improve or maintain cognitive ability in individual with cognitive impairment. These therapies can be categorized into four general approaches: (1) cognition-oriented treatments (e.g., reality orientation, skills training), (2) emotion-oriented treatments (e.g., supportive therapy, validation/integrated emotion-oriented care, Snoezelen, reminiscence), (3) behaviour-oriented treatments (behaviour therapy), and (4) stimulation-oriented treatments (e.g., activity or recreational therapy, art therapy, music therapy, exercise, psychomotor therapy). See, e.g., Carrion et al (2018) Dementia and Geriatric Cognitive Disorders 46:1 and Guidelines from the American Psychiatric Association.

In some embodiments, the presently described biomarker panel is used in combination with cognitive therapy, e.g., to determine effectiveness of the therapy or to slow cognitive decline.

In some embodiments, a spinal tap can be ordered for an individual to obtain cerebrospinal fluid (CSF). Measuring amyloid (e.g., Aβ-42) and/or tau (e.g., total tau and phosphorylated tau) levels in CSF can be useful for confirming a result from the presently described cognitive impairment biomarker panel, as these are associated with plaque formation in the brains of AD patients.

Brain imaging can be used for diagnosis of cognitive impairment because neurodegeneration often parallels and precedes the cognitive decline that is symptomatic of AD. The four types of imaging modalities are structural MRI, functional MRI, ¹⁸F-2-fluoro-2-deoxy-D-glucose (FDG) PET, and amyloid-PET. Structural or compositional abnormalities can be monitored with MRI scans, while FDG-PET monitors glucose metabolism mechanisms to identify areas of decreased brain activity. Of the various imaging methods, amyloid-PET is the most reliable diagnostic imaging tool because of its ability to characterize aggregated Aβ within the brain by utilizing amyloid tracers. Although imaging biomarkers are approved for clinical use and are considered advantageous due to their reliability in accurate diagnoses, the economic burden and accessibility issues associated with these imaging modalities continue to impede their comprehensive use in identifying AD. In addition to these difficulties, MRI and FDG-PET scans often struggle to distinguish AD from other neurodegenerative disorders. These techniques can therefore be advantageously combined with the presently described cognitive impairment biomarker panel, e.g., if an elevated result is returned for an individual.

In some embodiments, the methods described herein include administering a treatment to an individual that is predicted to develop or has a cognitive disorder, e.g., as determined by an elevated cognitive impairment biomarker profile. Alzheimer's and cognitive impairment are not fully curable, certain drug options are available and under development that address symptoms. These include certain anti-amyloid antibodies (e.g. aducanumab, gantenerumab, solanezumab), cholinesterase inhibitors, memantine verubecestat, lanabecestat, heteroaryl carboxamides (see U.S. Pat. No. 10,487,079), metabolites of (1R-trans)-N-[[2-(2,3-dihydro-4-benzofuranyl)cyclopropyl]methyl]propenamide (see U.S. Pat. No. 9,617,203), Pyrrolo[3,2-d]pyrimidine-2,4(3H,5H)-dione derivatives (see U.S. Pat. No. 9,440,983). More information on treatments for AD and cognitive impairment can be found at the website alz.org.

Further included are uses for treatments such as those listed above for treating cognitive impairment or AD in light of information revealed by the cognitive impairment biomarker panel as described herein. Such treatments can also be used in the manufacture of a medicament for treating cognitive impairment or AD in light of information revealed by the cognitive impairment biomarker panel as described herein.

The following non-limiting examples are provided to illustrate the present invention and in no way limit the scope thereof.

### EXAMPLES

We sought to focus on microRNA (miRNA) robustly detected in plasma at specific stages of cognitive decline, which required standardization of miRNA isolation and analytical methods. We sought to identify robust microRNA-based biomarker panels reflective of the progression of cognitive impairment and Alzheimer's disease (AD).

There was significant variation in the reported miRNAs associated with AD in the literature, as shown in Table 2. Table 2 also shows the results of our studies, obtained using standardized sample types and methods, alongside those reported in the literature.

TABLE 2
Study results. Shows the results of our studies, obtained using standardized
sample types and methods, alongside those reported in the literature. Source
of biological samples used as well as direction of expression are shown.

| Guévremont et al. | | | | Literature | | |
|---|---|---|---|---|---|---|
| microRNA | Aβ+ | MCI | AD | Source | Direction | Reference |
| miR-27a-3p | up | up | down | CSF | down | Sala Frigerio et al 2013 |
| | | | | Blood | down | Su et al 2019 |
| | | | | Brain | up | Cogswell et al 2008 |
| | | | | Brain | n/a | Mallick et al 2011 |
| | | | | Brain | down | Wang et al 2011 |
| | | | | Brain | up | Lau et al 2013 |
| | | | | Brain | up | Pogue et al 2018 |
| miR-27b-3p | up | up | down | BDM | up | Guedes et al 2016 |
| | | | | CSF | down | Lusardi et al 2017 |
| | | | | Serum | down | Agostini et al 2019 |
| | | | | Brain | up | Cogswell et al 2008 |
| | | | | Brain | down | Wang et al 2011 |
| | | | | Brain | n/a | Mallick et al 2011 |
| miR-29c-3p | up | up | nc | Serum | down | Jia et al 2016 |
| | | | | Serum | down | Wu et al 2017 |
| | | | | CSF | down | Gui et al 2015 |
| | | | | CSF | down | Sorensen et al 2016 |
| | | | | Blood | down | Yang et al 2015 |
| | | | | Brain | nc | Hebert et al 2008 |
| | | | | Brain | down | Wang et al 2011 |
| | | | | Brain | up | Zong et al 2011 |
| | | | | Brain | down | Lei et al 2015 |
| | | | | Brain | down | Su et al 2019 |
| miR-122-5p | down | up | up | Serum | down | Kumar et al 2017 |
| | | | | Brain | up | Satoh et al 2012 |
| miR-142-3p | up | down | down | Plasma | down | Kumar et al 2013 |
| | | | | Plasma | down | Cosin-Tomas et al 2017 |
| | | | | Plasma | down | Nagaraj et al 2017 |
| | | | | Serum | up | Denk et al 2018 |
| | | | | CSF | down | Lusardi et al 2017 |
| | | | | CSF | up | Wiedrick et al 2019 |
| | | | | Brain | up | Lau et al 2013 |
| miR-148a-3p | up | up | up | Blood | down | Satoh et al 2015 |
| | | | | Brain | up | Cogswell et al 2008 |
| miR-193b-3p | down | nc | up | Blood | down | Liu et al 2014 |
| | | | | Serum | down | Yang et al 2018 |
| | | | | CSF | down | Liu et al 2014 |
| | | | | Brain | down | Wang et al 2011 |
| miR-195-5p | up | up | up | Serum | up | Wu et al 2017 |
| | | | | CSF | down | Cogswell et al 2008 |
| | | | | CSF | down | Lusardi et al 2017 |
| | | | | CSF | up | Wiedrick et al 2019 |
| | | | | Brain | down | Sun et al 2015 |
| | | | | Brain | down | Zhu et al 2012 |
| | | | | Brain | up | Zhang et al 2016 |
| | | | | Brain | up | Lau et al 2013 |
| miR-324-5p | up | down | down | Brain | up | Cogswell et al 2008 |
| | | | | Brain | n/a | Mallick et al 2011 |
| miR-335-5p | up | up | up | Serum | up | Cheng et al 2015 |
| | | | | CSF | down | Denk et al 2015 |
| | | | | Brain | up | Cogswell et al 2008 |
| | | | | Brain | down | Moradifard et al 2018 |
| miR-885-5p | down | up | up | Serum | down | Tan et al 2014 |

### Cohorts and Blood Procedures

Otago Alzheimer's disease (Otago-AD): Participants were recruited from the Otago area. Medical records were reviewed and the diagnosis of probable AD was made by the consensus decision of a consultant neurologist and clinical psychologist. Individuals were classified as AD participants (n=44) if they meet the National Institute of Neurological and Communicative Disorders and Stroke and the Alzheimer's Disease and Related Disorders Association (NINCDS-ADRDA; McKhann et al., 1984) criteria. The control group (n=49; age and sex matched) underwent the same in-depth neuropsychological testing and neurological assessments. All AD participants had neuroimaging (MRI or CT) with images and clinical records inspected for confounding comorbidities. Refer Table 3 for summary of blood processing procedures.

TABLE 3
Procedures used for handling and processing blood specimens for each cohort analysed in this study.

| | | | | | | Total Processing | | |
|---|---|---|---|---|---|---|---|---|
| | | | Centifuge | | | Time | Storage | |
| | | Collection | | Time | Temp | Blood collection | Volume | Temp |
| Cohort | Fasting | Tubes | Speed (g) | (min) | (° C.) | to final storage | (μl) | (° C.) |
| Otago-AD | Yes | EDTA | Step 1: 1200 | 15 | 4 | within 2 hours | 250 | −80 Freezer |
| | | tubes | s/n transfered to | 15 | 4 | | | |
| | | | sterile tubes on ice | | | | | |
| | | | Step 2: 1200 | | | | | |
| | | | plasma aliquoted | | | | | |
| PMed | Yes/no | EDTA | Step 1: 1200 | 10 | 20 | within 1 hour | 250 | −80 Freezer |
| | | tubes | s/n transfered to | | | | | |
| | | | sterile tubes and | | | | | |
| | | | aliquoted on ice | | | | | |
| AIBL | Yes | EDTA | Step 1: 200 | 10 | 20 | within 3.5 hours | 250-500 | Liquid |
| | | tubes | s/n transfered to | 15 | 20 | | | Nitrogen |
| | | | sterile tubes | | | | | |
| | | | Step 2: 800 | | | | | |
| | | | plasma aliquoted | | | | | |

ApoEε genotyping: Genomic DNA was extracted from white blood cells using a NucleoSpin Tissue XS kit (Macherey-Nagel) according to the manufacturer's instruction. ApoE-ε4 genotype was assessed through TaqMan genotyping assays (TaqMan SNPs; Rs429358/Rs7412; Life Technologies, Mulgrave, VIC, Australia).

MCI samples: Plasma samples were purchased from PrecisionMed Inc (Solano Beach, CA, USA). These included plasma from MCI (n=36) and control (n=40) age and sex matched participants. Diagnosis was based on Modified ADAS-Cog, CDRs, Wechsler Memory Scale and MMSE scores (deficits in >2 areas) alongside CT or MRI imaging.

Samples from Australian Imaging, Biomarker & Lifestyle Flagship Study of Ageing (AIBL): Plasma samples donated by individuals with probable AD (n=21), MCI (n=38), Aβ+ (n=21) and cognitively normal and Aβ− age and sex matched cognitively normal controls (n=20). Diagnosis was based on clinical assessment (Ellis et al., 2009. Using the centiloid scale, a global measure of amyloid burden, participants were defined as Aβ+ (>45 CL) or Aβ− (0-20 CL) (Rowe et al, 2018). The Aβ+ participants all converted to the MCI stage (n=21) and most of the MCI participants converted to the AD stage (n=18). ApoE genotyping was carried out as described (Gupta et al 2015).

The demographic details of the cohorts and longitudinal studies are shown in Table 4. Abbreviations are as follows: Participants: HC, cognitively normal control; Aβ+, cognitively normal amyloid positive; Aβ−, cognitively normal amyloid negative; MCI, mild cognitive impairment; AD, Alzheimer's disease; F, female; M, male; MMSE, Mini-Mental State Examination; ApoEε4, apolipoprotein Eε4; p-value: Student t-tests, compared to HC p<0.05.

TABLE 4
Demographic characterisation of cohorts.
Cross-sectional study

| Otago-AD Cohort | HC | AD | p-value | PMed Cohort | HC | MCI | p-value |
|---|---|---|---|---|---|---|---|
| Number | 49 | 44 | | Number | 40 | 36 | |
| Age (mean ± SD) | 74.0 ± 5.9 | 75.5 ± 8.3 | 0.332 | Age (mean ± SD | 71.5 ± 3.9 | 73.2 ± 5.0 | 0.109 |
| Sex (F:M) | (25:24) | (23:21) | 0.905 | Sex (F:M) | (25:24) | (18:18) | 1 |
| MMSE (mean ± SD) | 28.7 ± 1.1 | 19.0 ± 6.2 | <0.0001 | MMSE (mean ± SD) | 29.6 ± 0.7 | 24.9 ± 1.4 | <0.0001 |
| ApoE_{ε}4−:ApoE_{ε}4+ (%) | 73:27 | 40:60 | 0.003 | ApoE_{ε}4−:ApoE_{ε}4+ (%) | n/a | n/a | |

| AIBL cohort | HC | Aβ+ | p-value | MCI | p-value | AD | p-value |
|---|---|---|---|---|---|---|---|
| Number | 20 | 21 | | 38 | | 21 | |
| Age (mean ± SD) | 74.7 ± 6.4 | 74.3 ± 5.7 | 0.851 | 75.8 ± 6.2 | 0.448 | 75.9 ± 7.8 | 0.616 |
| Sex (F:M) | (10:10) | (11:10) | 0.883 | (17:21) | 0.711 | (10:11) | 0.883 |
| MMSE (mean ± SD) | 29.3 ± 0.9 | 28.6 ± 1.3 | 0.989 | 26.4 ± 3.2 | <0.0001 | 19.5 ± 6.2 | <0.0001 |
| Image.PET.Centiloid | −0.5 ± 5.9 | 70.0 ± 29.8 | <0.0001 | 83.8 ± 33.2 | <0.0001 | 102.2 ± 23.1 | <0.0001 |
| (mean ± SD) | | | | | | | |
| ApoE_{ε}4−: ApoE_{ε}4+ (%) | 80:20 | 33:67 | <0.0001 | 32:68 | <0.0001 | 29:71 | <0.0001 |

Longitudinal study

| AIBL cohort | Aβ+/MCI | MCI/AD |
|---|---|---|
| Number | 21 | 18 |
| Age (mean ± SD) | (74.3 ± 5.7):(77.9 ± 5.5) | (74.7 ± 7.1):(77.6 ± 6.9) |
| Sex (F:M) | (11:10) | (8:10) |
| MMSE (mean ± SD) | (28.6 ± 1.3):(26.4 ± 1.5) | (26.3 ± 3.1):(23.3 ± 3.4) |

Abbreviations: Participants: HC, cognitively normal control; Aβ+, cognitively normal amyloid positive; Ab−, cognitively normal amyloid negative; MCI, mild cognitive impairment; AD, Alzheimer's disease; F, female; M, male; MMSE, Mini-Mental State Examination, ApoEε4, apolipoprotein Eε4; p-value: Student t-tests, compared to HC; p < 0.05.

### MicroRNA Expression Profiling

MicroRNA expression profiling was standardized using TaqMan microfluidics arrays. RNA was isolated from plasma using MirVana Paris (Life Technologies, Cat #AM1556M) following comparison of three different extraction protocols (TRIzol/Norgen, MirVana, Norgen). Following an initial screen of 784 microRNA using standard TaqMan microfluidics arrays (A and B cards), we created custom-designed microfluidics arrays representing 186 microRNA highly detected in plasma, or highly correlated with neurological disease and controls (U6 snRNA and ath-miR-159a). This approach was successfully used in our previous work assessing microRNA levels in plasma during aging and development of amyloidosis in the APP/PS1 transgenic mouse model (Ryan 2018).

A fixed volume (3 μl) of total RNA (˜50 ng) was converted to complementary-DNA (cDNA) using custom Megaplex RT human primer pool (Applied Biosystems) and TaqMan microRNA reverse transcription kits. cDNA was pre-amplified (12 cycles) using custom Megaplex PreAmp human primers Pool before qPCR (Automatic baseline threshold; ViiA-7 real-time PCR instrument, Quantstudio Real-Time PCRv1.3 Software; Applied Biosystems). Raw Ct values analysis was performed using the Bioconductor HTqPCR package version 1.10.0 (Dvinge et al, 2009) in computational environment R version 3.3.4. MicroRNA which were not expressed in all samples or had Ct<12 and >33 were excluded. All samples passed the miR-23a/miR-451 test of hemolysis (Blondal et al., 2013).

### Statistical Analysis

Cross-sectional studies: Following data normalization using Norm Rank Invariant, differentially expressed microRNA (case/matched cohort control; p<0.05) were identified using empirical-Bayes moderated t-tests and p-values adjusted using the Benjamini and Hochberg procedure to control for false discovery. Outliers were identified using Grubb's test. Normal distribution of the data was confirmed using the D'Agostino and Pearson omnibus normality test (p>0.05). Data were processed and the heatmap generated using GraphPad Prism (Version 8).

Meta-analyses were conducted using the R package “metafor” (Version 2.0-0; available at CRAN.R-project.org/package=metafor). A fixed-effect model was chosen for Aβ+ group, while a random-effect model was chosen for the MCI and AD groups (DerSimonian and Laird method). Estimate heterogeneity between studies was assessed using Cochran's Q and the I² statistic; where Qep<0.1; I² (%) >75% was considered significantly heterogeneous in the AD group. Results were visualized with Forest Plots, showing the the pooled effect size estimate, along with their confidence intervals (95% CI).

Univariate and multivariate logistic regressions were performed with the Forward: Wald method (MedCalc, Version 15.11.4). The goodness of fit for each logistic regression model was evaluated using the Hosmer-Lemeshow test (p>0.05). ROC Area Under the Curve (AUC) were evaluated for the overall model fit (p<0.05). Log-rank test were performed using the Mantel-Cox method (GraphPad Prism Version 8) to compare expression (normalized Ct) of the diseased and control groups; p<0.05 was considered significant.

Bioinformatics analysis: DIANA-microT v3.0 (Tarbase v7.0) and miRTarBase (release 7.0), using the most stringent algorithm parameters, were employed to identify validated targets of the 16 candidate biomarker miRNAs. Using DAVID 9 v6.7) (http://david.ncifcrf.gov), we focused on genes expressed in brain and blood. Biological pathways enriched within this group were identified using the Enrichr tool (see the website at amp.pharm.mssm.edu/Enrichr) to search the user-curated Wikipathways. Kegg Mapper (https://www.genome.jp/kegg/mapper.html) was used to colour the genes associated with each disease state.

Longitudinal studies: To identify change in microRNA expression with disease progression, Kaplan—Meier plots (GraphPad Prism, Version 8) were constructed using a subgroup of longitudinal samples from the AIBL cohort (Aβ+ to MCI convertors, n=21; MCI to AD convertors, n=18). Log-rank tests were used to compare the distributions of each microRNA expression (normalized Ct) and determine if their expression correlates with progression of the disease from Aβ+ to AD; median and 95% CI are reported; p<0.05 was considered significant. AUC were determined as above; 95% CI are reported.

Generalized estimating equation (SPSS, version 25.0) were used to determine significant effects in the longitudinal samples. The dependent variable was the microRNA expression studied (normalised Ct). Compound symmetry was used for the working correlation matrix structure and the Wald chi-square tested for the effect of group, followed by pairwise comparisons of the estimated marginal means at each set. The mean difference is significant at the 0.05 level.

Pearson's correlation coefficient r with P-value were generated for multiple variables, including normalised Ct, that could possibly explain the different expression in microRNA using method using MedCalc software Version 15.11.4.

Bioinformatics analysis: DIANA-microT v3.0 (Tarbase) and miRTarBase (release 7.0), using the most stringent algorithm parameters, were employed to identify validated targets of the 16 candidate biomarker miRNAs. Using DAVID (v6.7) (http://david.ncifcrf.gov), we focused on genes expressed in brain and blood. Biological pathways enriched within this group were identified using the Enrichr tool (http://amp.pharm.mssmedu/enrichr) to search the user-curated wikipathways. Kegg Mapper (https://www.genome.jp/kegg/mapper.html) was used to colour the genes associated with each disease stage.

### Characterization of Cohorts

Cohort demographic and clinical features of the cross-sectional and longitudinal cohorts utilized in our studies are summarized in Table 4. For the Otago-Alzheimer's disease study (Otago-AD), we recruited a volunteer cohort (n=93) of AD and age and sex-matched healthy control (HC) participants from the Otago area. Significant differences between the AD and HC were found for MMSE score (t(83)=9.647, p<0.0001) and ApoE genotype (t(86)=−3.556, p=0.003). We found that 60 % of AD participants were carriers of the ApoEε4 allele (Odds ratio: 4.6203 (95% CI 1.85-11.57), a value highly consistent with previous literature. We also obtained plasma samples and associated demographic data from Precision Med (PMed; Solano Beach, USA) and The Australian Imaging Biomarkers and Lifestyle study (AIBL). The PMed cohort (n=76) consisted of plasma samples given by individuals with MCI as well as age and sex-matched HC participants. Significant differences between the PMed MCI and HC groups were found for MMSE score (t(74)=−18.872, p<0.0001); ApoE genotype was not available. The AIBL cohort (n=100) consisted of plasma samples donated by individuals with MCI, AD as well as age and sex-matched HC participants who were either determined to be amyloid negative (Aβ−) or amyloid positive (Aβ+; centiloid scale: (−) 0-20CL ; (+)>45CL; Table 4). All MCI and AD participants were Aβ+. Significant differences in both MMSE score and ApoE genotype were found between the HC groups and MCI (t(56)=-6.603, p <0.001; (t(39)=4.314, p<0.0001) or AD (t(39)=−6.990, p<0.001; t(56)=4.411,p<0.001). We found that that 68% of MCI and 76% of AD participants were carriers of the ApoEε4 allele (Odds ratio: MCI: 12.28 (95% CI 3.00-50.01); AD: 14.17 (3.04-66.75)).

### Identification of Differentially Expressed microRNA: Cross-Sectional Analysis

Despite many studies, it appears that heterogeneity within the study cohorts, blood processing and miRNA analysis protocols have hindered identification of miRNA-based biomarker panels for prognosis or diagnosis of AD (meta; O'Bryant). We reasoned that in order for a biomarker to be clinically relevant, it should be independent of cohort-cohort variation, but may vary according to biofluid or method chosen for miRNA analysis. Further, based on our previous work where we showed that specific miRNAs vary in expression during the development of amyloidosis, we reasoned that the expression of specific miRNA will vary with the progression of AD. Accordingly, to identify miRNA-based biomarker panels relevant across cohorts, but specific to disease stage, we used qPCR TaqMan microfluidics arrays to quantify microRNA in plasma from within Aβ+, MCI (PMed, AIBL) and AD (Otago-AD, AIBL) cohorts, relative to their respective HCs. Differentially expressed miRNA were identified according to the following three criteria: Fold change (FC) ±0.2, empirical-Bayes moderated t-tests p<0.05 and expressed in all samples. miRNA that were found to be significantly differentially expressed within at least one group (Table 1 Fold Change).

This mode of analysis showed considerable similarities in the miRNA altered in the AD cohorts, with 75% being altered in the same direction (59% up; 16% down). It appears that the miRNA that were not validated between the AD cohorts were largely heterogeneous in the MCI group as well. Indeed, the MCI group overall showed greater heterogeneity with only about 50% of miRNA being altered in the same direction (39% up; 9% down). Interestingly, of the miRNA consistently upregulated in the MCI group, 62% were also upregulated in the Aβ+ group, and 75% of the miRNA regulated in the Aβ+ group were also consistently regulated in the AIBL-MCI group, giving weight to the conclusion that these miRNA are altered early in the progression of AD.

Focusing on the microRNA (n=32) which fulfilled the fold change criteria (FC±0.2), we found that miR-195-5p, a miRNA known to target the 3′UTR of BACE1 and reduced in AD post-mortem brain, was consistently upregulated across all cohorts and disease groups. Further, miR-885-5p was shown to be downregulated in the Aβ+ group, yet consistently upregulated in all the MCI and AD groups.

Within the AD groups 10 microRNA appear to be consistently upregulated (miR-122-5p, miR-132-3p, miR-193b-3p, miR-195-5p, miR-320-3p, miR-365-3p, miR-378-3p, miR-486-3p, miR-532-5p, miR-885-5p) and 5 downregulated (miR-27a-3p, miR-27b-3p, miR-142-3p, miR-324-5p, and miR-652-3p,).

Within the MCI groups 9 microRNA are consistently upregulated (miR-27a-3p, miR-27b-3p, miR-132-3p, miR-148a-3p, miR-195-5p, miR-199a-3p, miR-335-5p, miR-483-5p, miR-885-5p,) and one consistently downregulated (miR-142-3p).

Together these analyses show that while holding the blood fraction and analysis platform constant, it is possible to identify consistent alterations in miRNA expression patterns across independent cohorts and within different disease stages.

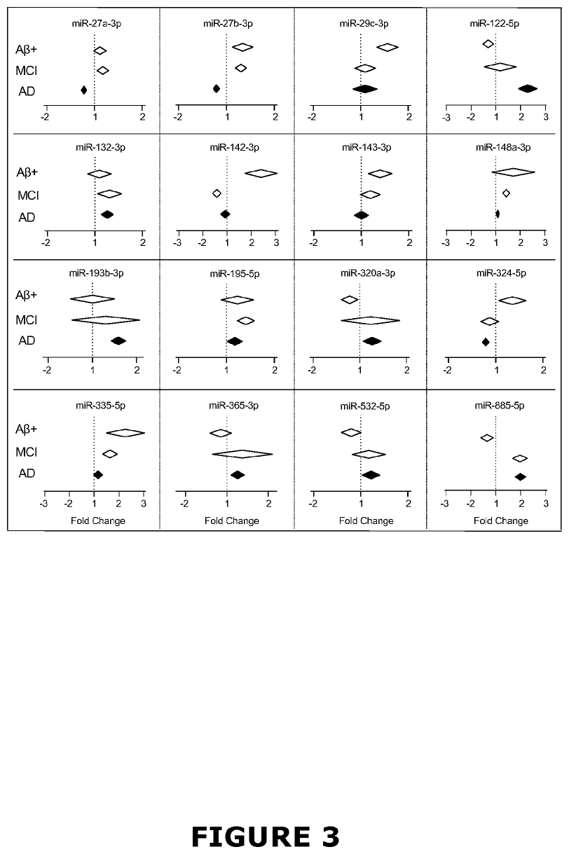

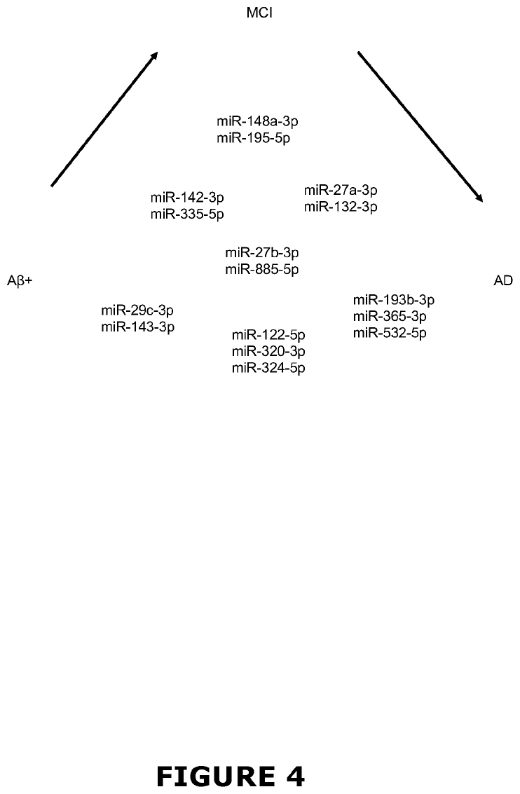

To further interrogate these findings, we pooled the data between like cohorts by meta-analysis to estimate a combined effect, and increase our statistical power. Meta-analytical models were fitted and the weighted fold change (pooled estimated effect size) of each miRNA (FIG. 3 and Table 5) and their 95% confidence intervals were obtained. Filtering the data by significant heterogeneity in the AD group (Qep test, I² statistics) resulted in a set of 16 putative biomarker microRNA (FIG. 3). The Venn Diagram (FIG. 4) summarises the association of specific microRNA and disease stage, with miR-27b-3p and miR-885-5p significantly regulated in all disease groups. Notably, these miRNA also show low heterogeneity (FIG. 3). Drawing on data from three independent cohorts, these analyses confirm that specific miRNA are dynamically expressed across the Aβ+, MCI or AD groups relative to HCs and that a unified biomarker signature is obtainable.

TABLE 5
Output of meta-analyses and heterogeneity tests.

| | | | Main Observed outcome | | | | | Test of | |
|---|---|---|---|---|---|---|---|---|---|
| | # Disease/ | | Mean estimate | | | | | Heterogeneity | |
| miRNA | controls | Stage | (Fold Change ± SE) | 95% CI | zval | pval | var | Qep | I² |
| miR-27a-3p | 21/20 | AB+ | 1.11 ± 0.07 | −1.02 to −1.24 | 16.90 | <0.0001 | 0.004 | 1 | 0 |
| | 74/60 | MCI | 1.17 ± 0.06 | 1.05 to −1.30 | 18.95 | <0.0001 | 0.004 | 0.19 | 42.1 |
| | 65/69 | AD | −1.28 ± 0.03 | −1.37 to −1.20 | 29.29 | <0.0001 | 0.001 | 0.86 | 0 |
| miR-27b-3p | 21/20 | AB+ | 1.34 ± 0.11 | 1.12 to 1.55 | 12.24 | <0.0001 | 0.010 | 1 | 0 |
| | 74/60 | MCI | 1.30 ± 0.06 | 1.19 to 1.41 | 22.79 | <0.0001 | 0.003 | 0.83 | 0 |
| | 65/69 | AD | −1.27 ± 0.03 | −1.37 to −1.18 | 25.08 | <0.0001 | 0.001 | 0.92 | 0 |
| miR-29c-3p | 21/20 | AB+ | 1.56 ± 0.11 | 1.34 to 1.79 | 13.74 | <0.0001 | 0.010 | 1 | 0 |
| | 74/60 | MCI | 1.09 ± 0.11 | −1.14 to 1.31 | 9.94 | <0.0001 | 0.010 | 0.25 | 23.2 |
| | 65/69 | AD | 1.03 ± 0.04 | −1.05 to 1.11 | 25.32 | <0.0001 | 0.002 | 0.43 | 0 |
| miR-122-5p | 21/20 | AB+ | −1.45 ± 0.11 | −2.13 to −1.10 | 6.20 | <0.0001 | 0.010 | 1 | 0 |
| | 74/60 | MCI | 1.28 ± 0.38 | −2.33 to 2.00 | 3.37 | 0.0007 | 1.140 | 0.11 | 85.7 |
| | 65/69 | AD | 2.29 ± 0.19 | 1.91 to 2.67 | 11.84 | <0.0001 | 0.040 | 0.23 | 29.0 |
| miR-132-3p | 21/20 | AB+ | 1.09 ± 0.12 | −1.18 to 1.34 | 8.73 | <0.0001 | 0.020 | 1 | 0 |
| | 74/60 | MCI | 1.31 ± 0.13 | 1.05 to 1.56 | 10.12 | <0.0001 | 0.020 | 0.10 | 62.3 |
| | 65/69 | AD | 1.26 ± 0.06 | 1.14 to 1.38 | 20.01 | <0.0001 | 0.004 | 0.57 | 0 |
| miR-142-3p | 21/20 | AB+ | 2.40 ± 0.34 | 1.74 to 3.07 | 7.09 | <0.0001 | 0.120 | 1 | 0 |
| | 74/60 | MCI | −1.72 ± 0.09 | −2.44 to to 1.37 | 6.59 | <0.0001 | 0.010 | 0.19 | 90.6 |
| | 65/69 | AD | −1.43 ± 0.23 | −3.57 to 1.18 | 3.16 | 0.002 | 0.050 | 0.18 | 44.9 |
| miR-143-3p | 21/20 | AB+ | 1.40 ± 0.13 | 1.15 to 1.64 | 11.09 | <0.0001 | 0.020 | 1 | 0 |
| | 74/60 | MCI | 1.19 ± 0.10 | −1.02 to 1.40 | 11.31 | <0.0001 | 0.010 | 0.07 | 69.2 |
| | 65/69 | AD | −1.02 ± 0.09 | −1.27 to 1.17 | 10.30 | <0.0001 | 0.010 | 0.19 | 39.4 |
| miR-148a3p | 21/20 | AB+ | 1.70 ± 0.45 | −1.19 to 2.60 | 3.83 | <0.0001 | 0.200 | 1 | 0 |
| | 74/60 | MCI | 1.43 ± 0.07 | 1.29 to 1.57 | 20.06 | <0.0001 | 0.010 | 0.81 | 0 |
| | 65/69 | AD | 1.08 ± 0.04 | 1.01 to 1.15 | 29.99 | <0.0001 | 0.001 | 0.91 | 0 |
| miR-193b-3p | 21/20 | AB+ | 1.00 ± 0.26 | −2.00 to 1.51 | 3.91 | <0.0001 | 0.070 | 1 | 0 |
| | 74/60 | MCI | 1.30 ± 0.39 | −1.89 to 2.07 | 3.32 | <0.0001 | 0.150 | 0.01 | 87.0 |
| | 65/69 | AD | 1.59 ± 0.08 | 1.43 to 1.75 | 19.28 | <0.0001 | 0.010 | 0.92 | 0 |
| miR-195-5p | 21/20 | AB+ | 1.23 ± 0.17 | −1.12 to 1.56 | 7.18 | <0.0001 | 0.030 | 1 | 0 |
| | 74/60 | MCI | 1.40 ± 0.09 | 1.23 to 1.80 | 15.56 | <0.0001 | 0.010 | 0.26 | 20.3 |
| | 65/69 | AD | 1.20 ± 0.15 | 1.00 to 1.50 | 7.78 | <0.0001 | 0.020 | 0.1 | 56.5 |
| miR-320a-3p | 21/20 | AB+ | −1.28± 0.09 | −1.64 to −1.04 | 8.86 | <0.0001 | 0.010 | 1 | 0 |
| | 74/60 | MCI | 1.23 ± 0.32 | −1.64 to 1.86 | 3.88 | 0.0001 | 0.100 | <0.0001 | 98.3 |
| | 65/69 | AD | 1.26 ± 0.09 | 1.11 to 1.65 | 12.99 | <0.0001 | 0.020 | 0.55 | 0 |
| miR-324-5p | 21/20 | AB+ | 1.35 ± 0.15 | 1.07 to 1.64 | 9.24 | <0.0001 | 0.020 | 1 | 0 |
| | 74/60 | MCI | −1.18 ± 0.09 | −1.45 to 1.06 | 9.21 | <0.0001 | 0.010 | 0.11 | 59.5 |
| | 65/69 | AD | −1.28 ± 0.03 | −1.41 to −1.18 | 22.50 | <0.0001 | 0.001 | 0.43 | 0 |
| miR-335-5p | 21/20 | AB+ | 2.26 ± 0.40 | 1.49 to 3.04 | 5.72 | <0.0001 | 0.160 | 1 | 0 |
| | 74/60 | MCI | 1.64 ± 0.15 | 1.34 to 1.94 | 10.77 | <0.0001 | 0.020 | 0.18 | 45.4 |
| | 65/69 | AD | 1.16 ± 0.09 | −1.03 to 1.34 | 12.33 | <0.0001 | 0.060 | 0.20 | 38.2 |
| miR-365-3p | 21/20 | AB+ | −1.18 ± 0.13 | −1.69 to 1.11 | 6.47 | <0.0001 | 0.020 | 1 | 0 |
| | 74/60 | MCI | 1.43 ± 0.51 | −1.64 to 2.24 | 3.44 | 0.001 | 0.170 | <0.0001 | 91.4 |
| | 65/69 | AD | 1.26 ± 0.08 | 1.10 to 1.42 | 3.95 | <0.0001 | 0.010 | 0.55 | 0 |
| miR-532-5p | 21/20 | AB+ | −1.27 ± 0.11 | −1.69 to 1.00 | 7.48 | <0.0001 | 0.010 | 1 | 0 |
| | 74/60 | MCI | 1.17 ± 0.18 | −1.22 to 1.52 | 6.51 | <0.0001 | 0.030 | 0.01 | 86.9 |
| | 65/69 | AD | 1.22 ± 0.10 | 1.03 to 1.41 | 12.62 | <0.0001 | 0.010 | 0.90 | 0 |
| miR-885-5p | 21/20 | AB+ | −1.35 ± 0.13 | −2.04 to 1.01 | 5.77 | <0.0001 | 0.020 | 1 | 0 |
| | 74/60 | MCI | 1.97 ± 0.15 | 1.67 to 2.26 | 13.12 | <0.0001 | 0.020 | 0.86 | 0 |
| | 65/69 | AD | 1.99 ± 0.11 | 1.77 to 2.21 | 17.96 | <0.0001 | 0.010 | 0.36 | 0 |

Summary of effect sizes (mean pooled estimates) along with their confidence intervals (95% CI) Cohran's Q and the I² statistic were used to test for heterogeneity. Qep is a p-value for the test of (residual) heterogeneity with a p-value of < 0.05 indicating presence of heterogeneity. We have used a value of 0.10 as a cut-off for significance and a I² of <50% (Higgins et al. 2003). I² statistic is the percentage of observed total variation across studies that is due to the real heterogeneity and larger values show increasing heterogeneity.

As a method of prioritising candidate miRNA biomarkers from this 16-miRNA set, we next sought to rank the importance of the individual miRNAs using a statistical consensus approach (Wiedrick et al 2019 and Lusardi et al 2017), combining the output of the differential expression analysis (p-value), the distribution of normalised Ct values (log-rank p-value) and ability of the microRNA to predict membership of a disease group (AUC), refer Table 6.

TABLE 6
Consensus ranking of cohorts.

| Aβ+ | p-value (1) | | Rank | p-value | Rank | AUC | Rank | Sum of Rank | Final Rank | Aβ+ | Final Rank |
|---|---|---|---|---|---|---|---|---|---|---|---|
| miR-27a-3p | 0.592 | | 16 | 0.714 | 14 | 0.583 | 14 | 44 | 15 | miR-29c-3p | 1 |
| miR-27b-3p | 0.295 | | 12 | 0.055 | 6 | 0.726 | 2 | 20 | 6 | miR-355-5p | 2 |
| miR-29c-3p | 0.012 | | 3 | 0.002 | 1 | 0.755 | 1 | 5 | 1 | miR-885-5p | 3 |
| miR-122-5p | 0.025 | | 6 | 0.004 | 2 | 0.699 | 5 | 13 | 4 | miR-122-5p | 4 |
| miR-132-3p | 0.561 | | 15 | 0.782 | 15 | 0.548 | 15 | 45 | 16 | miR-142-3p | 5 |
| miR-142-3p | 0.019 | | 5 | 0.084 | 9 | 0.699 | 5 | 19 | 5 | miR-27b-3p | 6 |
| miR-143-3p | 0.027 | | 8 | 0.156 | 11 | 0.711 | 4 | 23 | 7 | miR-143-3p | 7 |
| miR-148a-3p | 0.220 | | 11 | 0.782 | 16 | 0.593 | 13 | 40 | 13 | miR-320a-3p | 8 |
| miR-193b-3p | 0.026 | | 7 | 0.316 | 12 | 0.674 | 8 | 27 | 10 | miR-532-5p | 9 |
| miR-195-5p | 0.535 | | 14 | 0.396 | 13 | 0.517 | 16 | 43 | 14 | miR-193b-3p | 10 |
| miR-320a-3p | 0.016 | | 4 | 0.152 | 10 | 0.669 | 10 | 24 | 8 | miR-324-5p | 11 |
| miR-324-5p | 0.298 | | 13 | 0.065 | 8 | 0.674 | 8 | 29 | 11 | miR-365-3p | 11 |
| miR-335-5p | 0.010 | | 2 | 0.005 | 3 | 0.726 | 2 | 7 | 2 | miR-148a-3p | 13 |
| miR-365-3p | 0.042 | | 10 | 0.063 | 7 | 0.629 | 12 | 29 | 11 | miR-195-5p | 14 |
| miR-532-5p | 0.048 | | 9 | 0.019 | 5 | 0.643 | 11 | 25 | 9 | miR-27a-3p | 15 |
| miR-885-5p | 0.005 | | 1 | 0.014 | 4 | 0.679 | 7 | 12 | 3 | miR-132-3p | 16 |
| MCI | p-value (1) | p-value (2) | Rank | p-value | Rank | AUC | Rank | Sum of Rank | Final Rank | MCI | Final Rank |
| miR-27a-3p | 0.845 | 0.045 | 15 | 0.168 | 4 | 0.527 | 10 | 29 | 10 | miR-142-3p | 1 |
| miR-27b-3p | 0.168 | 0.029 | 9 | 0.715 | 11 | 0.514 | 13 | 33 | 13 | miR-324-5p | 2 |
| miR-29c-3p | 0.856 | 0.595 | 16 | 0.367 | 7 | 0.514 | 14 | 37 | 15 | miR-195-5p | 3 |
| miR-122-5p | 0.352 | 0.593 | 14 | 0.567 | 8 | 0.520 | 12 | 34 | 14 | miR-148a-3p | 3 |
| miR-132-3p | 0.368 | 0.004 | 9 | 0.879 | 15 | 0.559 | 4 | 28 | 9 | miR-365-3p | 5 |
| miR-142-3p | 0.005 | 0.000 | 1 | 0.006 | 1 | 0.714 | 1 | 3 | 1 | miR-320a-3p | 6 |
| miR-143-3p | 0.043 | 0.664 | 11 | 0.868 | 13 | 0.555 | 5 | 29 | 10 | miR-193b-3p | 7 |
| miR-148a-3p | 0.016 | 0.000 | 2 | 0.070 | 3 | 0.521 | 11 | 16 | 3 | miR-885-5p | 7 |
| miR-193b-3p | 0.136 | 0.319 | 11 | 0.183 | 5 | 0.530 | 9 | 25 | 7 | miR-132-3p | 9 |
| miR-195-5p | 0.225 | 0.001 | 5 | 0.593 | 9 | 0.671 | 2 | 16 | 3 | miR-27a-3p | 10 |
| miR-320a-3p | 0.074 | 0.000 | 3 | 0.873 | 14 | 0.548 | 7 | 24 | 6 | miR-143-3p | 10 |
| miR-324-5p | 0.049 | 0.011 | 5 | 0.007 | 2 | 0.536 | 8 | 15 | 2 | miR-532-5p | 10 |
| miR-335-5p | 0.169 | 0.004 | 7 | 0.937 | 16 | 0.506 | 16 | 39 | 16 | miR-27b-3p | 13 |
| miR-365-3p | 0.288 | 0.002 | 7 | 0.733 | 12 | 0.587 | 3 | 22 | 5 | miR-122-5p | 14 |
| miR-532-5p | 0.478 | 0.007 | 13 | 0.657 | 10 | 0.551 | 6 | 29 | 10 | miR-29c-3p | 15 |
| miR-885-5p | 0.040 | 0.025 | 4 | 0.198 | 6 | 0.510 | 15 | 25 | 7 | miR-335-5p | 16 |
| AD | p-value (1) | p-value (3) | Rank | p-value | Rank | AUC | Rank | Sum of Rank | Final Rank | AD | Final Rank |
| miR-27a-3p | 0.041 | 0.016 | 1 | 0.003 | 2 | 0.765 | 1 | 4 | 1 | miR-27a-3p | 1 |
| miR-27b-3p | 0.026 | 0.530 | 4 | 0.007 | 5 | 0.722 | 2 | 11 | 2 | miR-27b-3p | 2 |
| miR-29c-3p | 0.147 | 0.582 | 8 | 0.062 | 7 | 0.504 | 16 | 31 | 9 | miR-122-5p | 2 |
| miR-122-5p | 0.001 | 0.938 | 7 | 0.002 | 1 | 0.644 | 3 | 11 | 2 | miR-193b-3p | 2 |
| miR-132-3p | 0.362 | 0.016 | 6 | 0.067 | 8 | 0.579 | 8 | 22 | 7 | miR-324-5p | 5 |
| miR-142-3p | 0.105 | 0.860 | 11 | 0.316 | 9 | 0.532 | 11 | 31 | 9 | miR-885-5p | 6 |
| miR-143-3p | 0.382 | 0.978 | 15 | 0.330 | 10 | 0.524 | 13 | 38 | 13 | miR-132-3p | 7 |
| miR-148a-3p | 0.857 | 0.966 | 16 | 0.671 | 13 | 0.530 | 12 | 41 | 14 | miR-365-3p | 8 |
| miR-193b-3p | 0.005 | 0.197 | 2 | 0.005 | 4 | 0.629 | 5 | 11 | 2 | miR-29c-3p | 9 |
| miR-195-5p | 0.706 | 0.844 | 13 | 0.779 | 14 | 0.515 | 15 | 42 | 15 | miR-142-3p | 9 |
| miR-320a-3p | 0.886 | 0.532 | 12 | 0.990 | 16 | 0.617 | 7 | 35 | 12 | miR-335-5p | 11 |
| miR-324-5p | 0.038 | 0.026 | 3 | 0.024 | 6 | 0.635 | 4 | 13 | 5 | miR-320a-3p | 12 |
| miR-335-5p | 0.440 | 0.381 | 10 | 0.669 | 12 | 0.533 | 10 | 32 | 11 | miR-143-3p | 13 |
| miR-365-3p | 0.037 | 0.871 | 8 | 0.340 | 11 | 0.544 | 9 | 28 | 8 | miR-148a-3p | 14 |
| miR-532-5p | 0.293 | 0.993 | 14 | 0.880 | 15 | 0.517 | 14 | 43 | 16 | miR-195-5p | 15 |
| miR-885-5p | 0.001 | 0.766 | 5 | 0.005 | 3 | 0.618 | 6 | 14 | 6 | miR-532-5p | 16 |

For each disease stage, each of the 16 miRNA identified in the meta-analysis were ranked using 3 independent criteria. The 3 rankings per miRNA were then summed to provide a final rank. Lower total rank sums resulted in highest rankings. The 3 ranking criteria were (1) differential expression (p-value; Table 1), (2) distribution of normalised Ct values (Log-rank tests; p-values) and (3) predictive power (AUC from logistic regression). (1) AIBL, (2) PMed, (3) Otago cohorts.

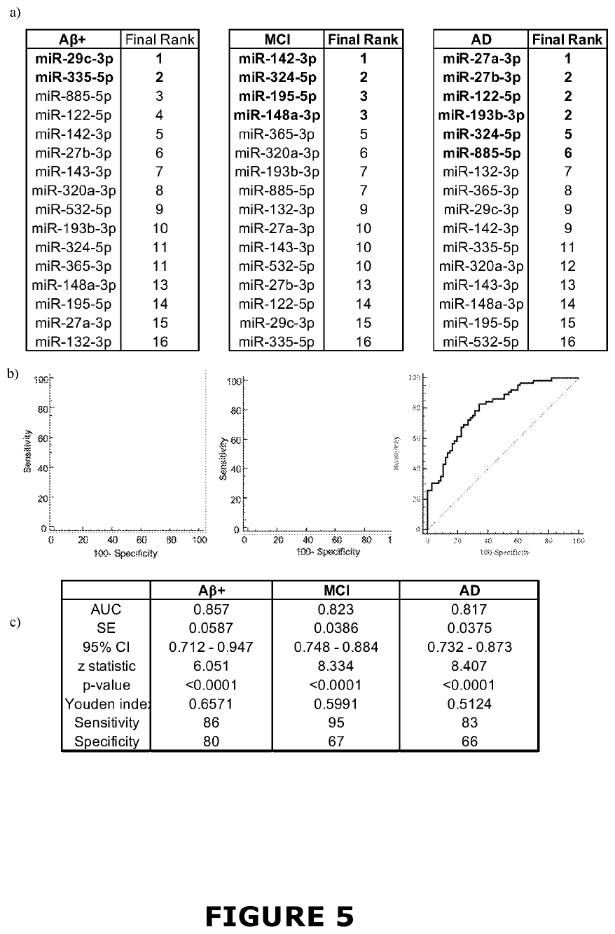

As shown in FIG. 5, a prioritized list of the putative biomarkers for each disease stage was obtained. In the AD group, the top ranked microRNA had the highest FC (Table 1). This was not true, however, for the Aβ+ or MCI groups, thus supporting the use this consensus approach. Next, we determined the ability of combinations of the top ranked microRNA to predict membership of the disease groups by ROC analysis. The number of microRNA were constrained to comply with the recommendation of Peduzzi et. al. (1996). Derived AUCs were Aβ+: 0.857 (miR-29c-3p and miR-335-5p); MCI: 0.823 (miR-142-3p, miR-324-5p, miR-195b-5p, miR-148a-3p) and AD: 0.817 (miR-27a-3p, miR-27b-3p, miR-122-5p, miR-193b-3p, miR-324-5p and miR-885-5p). This analysis suggests that unique microRNA biomarker signatures reflect each diseases stage and have prognostic and diagnostic utility.

### miRNA Expression: Longitudinal Analysis

To further explore the association of the miRNA biomarkers with disease progression we extracted a subgroup of longitudinal samples from the AIBL cohort comprising n=21 individuals who donated samples when classified as Aβ+ and MCI and n=18 individuals who donated samples when classified as MCI and AD (Table 4; FIG. 2).

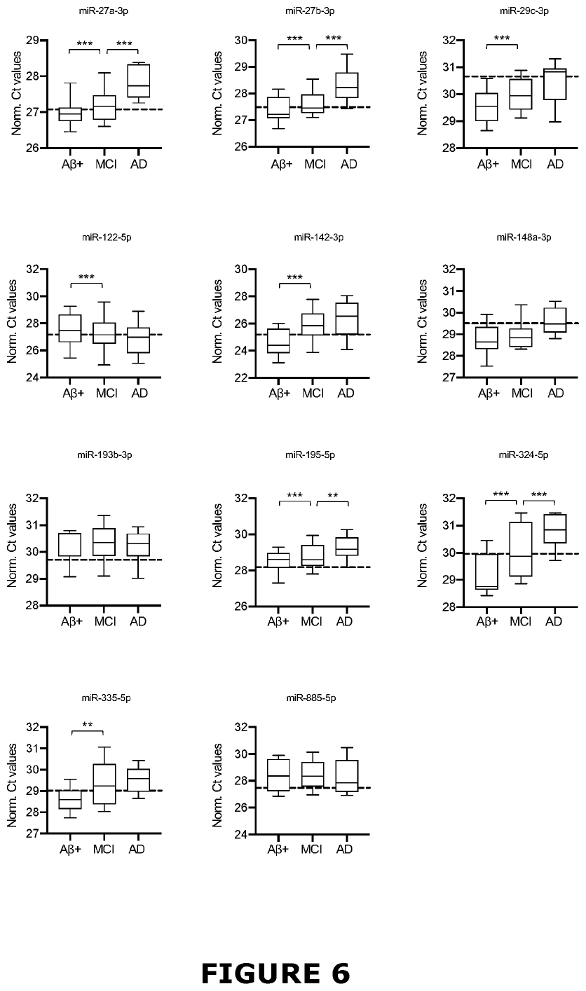

We assessed the trends in expression of our candidate biomarkers with the progression of disease using a generalised estimating equation (GEE, Table 7) to account for the longitudinal nature of the samples and the resulting lack of independence of the data sets. This analysis confirmed dynamic regulation of this group of microRNA within the individuals within this longitudinal cohort. Eight microRNA were shown to significantly alter in the transition from Aβ+ to MCI (up: miR-27a-3p, miR-27b-3p, miR-122-5p; down: miR-29c-3p, miR-142-3p, miR-195-5p, miR-324-5p, miR-335-5p) and four microRNA were shown to be significantly downregulated in the transition from MCI to AD (FIG. 6). This group included miR-27a-3p, miR-27b-3p, which were both upregulated in the Aβ+ to MCI transition, and miR-195-5p, miR-324-5p which were both downregulated in the MCI to AD transition. These results reinforce that alteration in microRNA levels occurs early in the disease and can be dynamic. However, this also shows that particular microRNA (such as miR-195-5p, miR-324-5p), are consistently regulated throughout the disease. In addition, these results show that the observed variation in microRNA levels are not simply a result of variation in preanalytical processing and indeed reflect the progression of disease.

TABLE 7
Output from generalized estimating equations (GEE).

| | | Estimated. | | | | p-value | | df = 2 (n = 78) | Overall |
|---|---|---|---|---|---|---|---|---|---|
| Dependent | Disease | Marginal | | 95% Wald CI | | Aβ+ vs | MCI vs | Wald | Model |
| Variable | Stage | means | SE | Lower | Upper | MCI | AD | Chi-Square | Sig. (p) |
| miR-27a-3p | Aβ+ | 27.30 | 0.05 | 27.21 | 27.35 | 0.00002 | 0.000006 | 43.13 | <0.00001 |
| | MCI | 27.10 | 0.03 | 27.04 | 27.17 | | | | |
| | AD | 27.54 | 0.08 | 27.40 | 27.69 | | | | |
| miR-27b-3p | Aβ+ | 27.83 | 0.07 | 27.70 | 27.96 | 0.00020 | 0.00022 | 31.58 | <0.00001 |
| | MCI | 27.56 | 0.05 | 27.47 | 27.65 | | | | |
| | AD | 28.03 | 0.10 | 27.83 | 28.22 | | | | |
| miR-29c-3p | Aβ+ | 29.72 | 0.07 | 29.57 | 29.86 | 0.000 | 0.169 | 19.84 | 0.000 |
| | MCI | 30.03 | 0.05 | 29.93 | 30.13 | | | | |
| | AD | 30.26 | 0.10 | 30.06 | 30.46 | | | | |
| miR-122-5p | Aβ+ | 27.62 | 0.09 | 27.44 | 27.80 | 0.0000 | 0.292 | 28.98 | 0.0120 |
| | MCI | 27.11 | 0.06 | 26.99 | 27.24 | | | | |
| | AD | 26.86 | 0.13 | 26.61 | 27.11 | | | | |
| miR-142-3p | Aβ+ | 24.99 | 0.16 | 24.68 | 25.30 | 0.0000 | 0.881 | 24.83 | 0.0000 |
| | MCI | 25.87 | 0.10 | 25.67 | 26.07 | | | | |
| | AD | 25.81 | 0.20 | 25.42 | 26.20 | | | | |
| miR-148a-3p | Aβ+ | 29.12 | 0.10 | 28.93 | 29.31 | 0.913 | 0.997 | 1.32 | 0.516 |
| | MCI | 29.01 | 0.06 | 28.89 | 29.13 | | | | |
| | AD | 29.08 | 0.11 | 28.85 | 29.30 | | | | |
| miR-193b-3p | Aβ+ | 30.45 | 0.10 | 30.24 | 30.65 | 0.415 | 0.642 | 3.74 | 0.154 |
| | MCI | 30.27 | 0.07 | 30.14 | 30.41 | | | | |
| | AD | 30.07 | 0.14 | 29.80 | 30.34 | | | | |
| miR-195-5p | Aβ+ | 28.10 | 0.10 | 27.91 | 28.30 | 0.000 | 0.003 | 30.47 | 0.133 |
| | MCI | 28.61 | 0.06 | 28.50 | 28.73 | | | | |
| | AD | 29.00 | 0.10 | 28.80 | 29.20 | | | | |
| miR-324-5p | Aβ+ | 29.37 | 0.10 | 29.17 | 29.57 | 0.0000 | 0.00009 | 42.98 | <0.00001 |
| | MCI | 29.95 | 0.07 | 29.81 | 30.08 | | | | |
| | AD | 30.61 | 0.14 | 30.34 | 30.87 | | | | |
| miR-335-5p | Aβ+ | 28.84 | 0.15 | 28.56 | 29.12 | 0.008 | 0.841 | 9.17 | 0.010 |
| | MCI | 29.35 | 0.09 | 29.18 | 29.53 | | | | |
| | AD | 29.30 | 0.16 | 28.99 | 29.61 | | | | |
| miR-885-5p | Aβ+ | 28.47 | 0.28 | 27.92 | 29.03 | 0.830 | 0.874 | 0.10 | 0.950 |
| | MCI | 28.40 | 0.21 | 27.99 | 28.80 | | | | |
| | AD | 28.33 | 0.32 | 27.70 | 28.97 | | | | |

Longitudinal samples were studied with GEE (generalize models for matched pairs; SPSS version 8). The dependent variable was the miRNA studied. Compound symmetry was used for the working correlation matrix structure and the Wald chi-square tested for the effect of group, followed by pairwise comparisons of the estimated marginal means at each Disease stage. The mean difference is significant at the 0.05 level. In bold are significant changes. Included in this table are the estimated marginal means of each model, the SE and 95% CI, the overall p-value for the model as well as the p-values for each group comparison.

Next, probing the diagnostic utility of the miRNA, we assessed the ability of each to predict membership of the disease groups by ROC analysis. Resulting AUCs ranged from 0.51 to 0.76, and pronouncedly increased to ranging from 0.8 to 0.95 by inclusion of ApoEε4 as a factor (Table 8). miR-29c-3p and miR-335-5p showed the strongest AUCs in the Aβ+ group, whereas, miR-142-3p, miR-148a-3p, and miR-27b-3p were strongest in the MCI group and miR-27a-3p and miR-27b-3p showed the strongest in the AD group. Furthermore, we found using Pearson correlations that amyloid-β load (centiloid values; Table 4) in the AD group was were significantly correlated with miR-27a-3p (r=0.466; p =0.002), miR-27b-3p (r=0.391; p=0.012) and miR-324-5p (r =0.406; p=0.009). Together these analyses suggest that these miRNA not only vary with the progression of AD, but may have prognostic utility and in particular highlight the association of miR-27a-3p, miR-27b-3p and miR-324-5p with amyloidosis.

TABLE 8
AIBL longitudinal cohort. Area Under the Curve (AUC) estimates
and 95% CI were used to establish the predictive power of miRNA
within the AIBL cohorts (AB+, MCI and AD, top to bottom). The
normalised Ct values were used to establish AUC ± ApoEε4 status.

| | AUC | 95% CI | AUC with ApoEε ± 4 | 95% CI |
|---|---|---|---|---|
| miR-27a-3p | 0.58 | 0.42-0.74 | 0.84 | 0.69-0.93 |
| miR-27b-3p | 0.73 | 0.57-0.85 | 0.86 | 0.72-0.95 |
| miR-29c-3p | 0.76 | 0.60-0.88 | 0.89 | 0.76-0.97 |
| miR-142-3p | 0.71 | 0.55-0.84 | 0.84 | 0.69-0.94 |
| miR-148a-3p | 0.59 | 0.43-0.74 | 0.82 | 0.67-0.93 |
| miR-193b-3p | 0.67 | 0.51-0.81 | 0.83 | 0.68-0.93 |
| miR-195-5p | 0.51 | 0.36-0.68 | 0.87 | 0.72-0.95 |
| miR-324-5p | 0.67 | 0.51-0.81 | 0.82 | 0.67-0.92 |
| miR-335-5p | 0.76 | 0.57-0.85 | 0.89 | 0.75-0.97 |
| miR-885-5p | 0.68 | 0.51-0.82 | 0.82 | 0.67-0.92 |
| miR-27a-3p | 0.67 | 0.44-0.70 | 0.83 | 0.71-0.96 |
| miR-27b-3p | 0.64 | 0.50-0.76 | 0.83 | 0.70-0.91 |
| miR-29c-3p | 0.55 | 0.41-0.68 | 0.85 | 0.73-0.93 |
| miR-142-3p | 0.64 | 0.50-0.76 | 0.87 | 0.75-0.94 |
| miR-148a-3p | 0.64 | 0.51-076 | 0.83 | 0.71-0.92 |
| miR-193b-3p | 0.62 | 0.49-0.75 | 0.79 | 0.67-0.89 |
| miR-195-5p | 0.58 | 0.44-0.71 | 0.84 | 0.74-0.93 |
| miR-324-5p | 0.57 | 0.44-0.70 | 0.85 | 0.74-0.93 |
| miR-335-5p | 0.62 | 0.48-0.74 | 0.83 | 0.71-0.92 |
| miR-885-5p | 0.65 | 0.51-0.77 | 0.80 | 0.67-0.90 |
| miR-27a-3p | 0.70 | 0.53-0.83 | 0.94 | 0.82-0.99 |
| miR-27b-3p | 0.66 | 0.51-0.80 | 0.93 | 0.81-0.99 |
| miR-29c-3p | 0.55 | 0.34-0.66 | 0.85 | 0.73-0.93 |
| miR-142-3p | 0.64 | 0.36-0.68 | 0.85 | 0.74-0.93 |
| miR-148a-3p | 0.54 | 0.37-0.69 | 0.85 | 0.70-0.94 |
| miR-193b-3p | 0.59 | 0.43-0.74 | 0.95 | 0.84-0.99 |
| miR-195-5p | 0.58 | 0.34-0.66 | 0.86 | 0.74-0.93 |
| miR-324-5p | 0.57 | 0.56-0.85 | 0.87 | 0.75-0.94 |
| miR-335-5p | 0.62 | 0.45-0.76 | 0.83 | 0.71-0.92 |
| miR-885-5p | 0.61 | 0.44-0.76 | 0.90 | 0.76-0.97 |

### Relationship between Putative Biomarker microRNA and Alzheimer's Disease: Biological Relevance

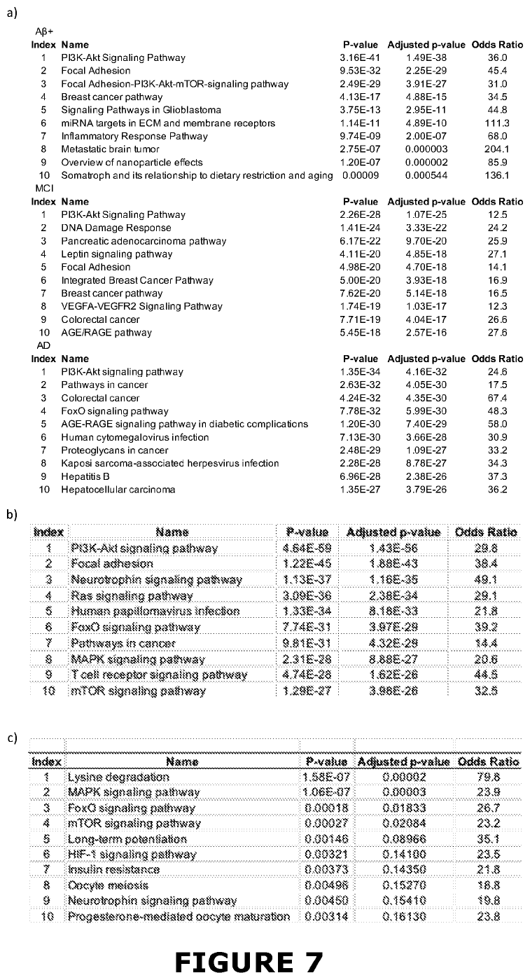

Using a bioinformatic approach we probed the association of the candidate microRNA biomarkers with AD molecular pathology. Focusing on the disease stage-related signatures (FIG. 5), we identified validated target mRNA and interpreted the resulting lists using the gene set enrichment tool, Enrichr (Kuleshov et al., 2016). Targets for the candidate Aβ+, MCI and AD biomarker microRNA groups were shown to be significantly enriched in AD-relevant Wikipathways, the highest overrepresented pathway identified for each of the disease stages (FIG. 7a) and in combination (FIG. 7b). Pathway analysis of all microRNA targets combined additionally identified Neurotrophin signalling, MAPK signalling and mTOR signalling. Neurotrophin signalling was also highlighted in pathway analysis of the microRNA correlated with centiloid values (FIG. 7c), alongside Insulin Resistance and Long-term potentiation. Alongside the observations that the candidate biomarker microRNA have previously been shown to be altered in either AD plasma and/or post mortem tissue (Table 2) this analysis reinforces the connection between the candidate microRNA biomarkers and the pathology underlying the progressive stages of AD.

### Discussion

Small noncoding RNA, in particular microRNA, are a central focus both as biomarkers of neurodegenerative diseases and novel therapeutic agents. To-date, there is no agreed consensus blood biomarker for AD, despite more than 30 studies proposing 100 candidate microRNA. This failure in replication may be due to inconsistent blood fraction methods and variation in microRNA analysis. By holding the blood fraction and mode of analysis constant together with a statistical consensus approach, we have determined a unique set of plasma microRNA associated with AD. Importantly, we have shown for the first time that plasma microRNA levels are altered before symptoms manifest and vary dynamically with disease progression. The individual microRNA which we have highlighted have all been previously associated with AD and using bioinformatics, we have shown that our candidate microRNA converge on PI3K-Akt signalling, a pathway with a well-established relationship with the molecular pathology underlying AD, including neurofibrillary tangles and microglial and astroglial inflammasome regulation. This lends weight to the conclusion that the microRNA compiled in our study comprise a valid set of AD-related biomarkers as well as reflect the disease processes occurring within the brain.

Our work particularly highlights elevated miR-29c-3p and miR-335-5p levels as novel biomarkers of early amyloidosis. Levels of both miR-29c-3p and miR-335-5p have previously been shown to be altered in AD biofluids (Table 2) and miR-29c-3p and BACE1 as well as miR-335-5p and Aβ levels are inversely correlated, suggesting that they both contribute directly to amyloid levels in the brain. Further, both miR-29c-3p and miR-335-5p have been shown to enhance memory performance in the Morris Water Maze. miR-335-5p is a neuronally-enriched microRNA and a proposed key regulator of AD-related gene networks and our bioinformatic analysis showed that together these microRNA map to Inflammatory Response and Glioblastoma as well PI3K and mTOR pathways. Indeed, miR-29c-3p is known to protect against inflammasome activation in microglia, suggesting a role in neuroprotection. Our finding that miR-335-5p is upregulated in plasma, supports the findings of Cheng et al., who showed this microRNA to be upregulated in extracellular vesicles isolated from plasma, from the AIBL-AD cohort. Interestingly, two previous studies have shown that miR-335-5p is downregulated in post-mortem brain, thus suggesting that AD is associated with an increase in the export of miR-335-5p into extracellular vesicles.

Within our biomarker signature we found that the expression of most microRNA varied across the progression of the disease. However, our cross-sectional study showed that miR-195-5p and miR-335-5p, were consistently higher than that of the control cohort but interestingly, in the AIBL longitudinal sub-group, we found that their levels were reducing with the progression of the disease, whilst remaining above control levels. Upregulation of miR-195-5p may also be part of a neuroprotective response as this microRNA inhibits both the expression of both BACE1 and APP and apoptosis. Further, knockdown of miR-195-5p has been shown to decrease dendritic length and number and the synaptically-located molecule, neurogranin (NRGN) is a validated target of miR-195-5p the levels of which are reduced in AD post-mortem tissue and neural derived exosomes.

Concomitant with change in the composition of the disease stage-related biomarker microRNA, we observed additional pathways mapping to the MCI and AD groups. In particular the MCI analysis, identified the AGE/RAGE Pathway and VEGFA-VEGFR2 signalling Pathway. This is interesting because both pathways are also linked to neuroprotection. Indeed, inhibition of advanced glycation end products and its receptor has been mooted as a potential AD therapy and miR-142, our most highly ranked microRNA in the MCI group, targets the RAGE pathway. As miR-142-3p directly targets inflammatory pathways, this leads to the suggestion that de-repression of miR-142-3p targets may be associated initially with curtailing a neuroinflammatory response. While there is much debate over the role of neuroinflammation in AD, it is intriguing that recently, a genetic variation in the promoter of miR-142 (rs2526377:A>G) which results in reduced expression, was shown to be significantly associated with a reduced risk of AD. Thus, further investigations into the role of miR-142 in AD are warranted.

The microRNA correlated with the amyloid load (centiloid values) in individuals with advanced AD mapped to the HIF-1 Signalling pathway. This pathway is interlinked with VEGF, MAPK and PI3K signalling and promotes amyloidogenic processing of APP. The plasticity-related pathways Neurotrophin Signalling and Long-term potentiation were also mapped to this group. miR-27a-3p, miR-27b-3p and miR-324-5p have previously been shown to be altered in blood or post-mortem brain tissue (refer Table 2). Indeed downregulation of miR-324-5p has been mooted to contribute to synaptic loss during aging, while miR-27b-3p is considered a proinflammatory microRNA, inhibiting expression tumour necrosis factor-α and interleukin-6. Interestingly, miR-27a-3p targets SERPINA3, which encodes a serine protease inhibitor associated with ApoE-ε4 genotype, inflammation and amyloid polymerization. Together these bioinformatic analyses highlight a strong relationship between out candidate biomarker microRNA inflammation and amyloidosis, lending weight to the conclusion that these plasma biomarkers reflect the disease processes occurring within the brain.

In summary, the plasma microRNA highlighted by our studies, derived by using a statistical consensus approach using multiple cohorts, vary with disease progression and reflect known steps underlying AD neuropathology and hence may be useful in disease risk prediction in clinical practice. Our early signature may be able to predict underlying pathology before individuals become symptomatic. These data are unique and need to be strengthened by further in-depth analysis of pre-symptomatic individuals and potentially by analysis of neuronal exosome-derived microRNA in plasma or CSF. It will also be important to understand the influence of other endophenotypes such as ApoE-ε4 status on the plasma levels of these microRNA as well as ethnicity of the study cohorts. These data will be highly valuable for improved criteria of inclusion into clinical trials where currently available cognitive behavioural and drug therapies can be further tested and thus the onset of disease can be delayed. That overall, we have shown that biomarkers are dynamic, altering with disease progression, emphases the need for longitudinal biomarker testing. The transition to our later signature may further identify at risk individuals and be useful in prioritising individuals for more advanced who warrant highly specialised testing.

It is not the intention to limit the scope of the invention to the abovementioned examples only. As would be appreciated by a skilled person in the art, many variations are possible without departing from the scope of the invention as set out in the appended claims.

### REFERENCES

Blondal, T., S. Jensby Nielsen, A. Baker, D. Andreasen, P. Mouritzen, M. Wrang Teilum and I. K. Dahlsveen (2013). “Assessing sample and miRNA profile quality in serum and plasma or other biofluids.” Methods 59(1): S1-6.

Chen, X., X. M. Jiang, L. J. Zhao, L. L. Sun, M. L. Yan, Y. Tian, S. Zhang, M. J. Duan, H. M. Zhao, W. R. Li, Y. Y. Hao, L. B. Wang, Q. 3. Xiong and J. Ai (2017). “MicroRNA-195 prevents dendritic degeneration and neuron death in rats following chronic brain hypoperfusion.” Cell Death Dis 8(6): e2850.

Ellis, K. A., A. I. Bush, D. Darby, D. De Fazio, J. Foster, P. Hudson, N. T. Lautenschlager, N. Lenzo, R. N. Martins, P. Maruff, C. Masters, A. Milner, K. Pike, C. Rowe, G. Savage, C. Szoeke, K. Taddei, V. Villemagne, M. Woodward and D. Ames (2009). “The Australian Imaging, Biomarkers and Lifestyle (AIBL) study of aging: methodology and baseline characteristics of 1112 individuals recruited for a longitudinal study of Alzheimer's disease.” Int Psychogeriatr 21(4): 672-687.

Gupta, V. B., J. D. Doecke, E. Hone, S. Pedrini, S. M. Laws, M. Thambisetty, A. I. Bush, C. C. Rowe, V. L. Villemagne, D. Ames, C. L. Masters, S. L. Macaulay, A. Rembach, S. R. Rainey-Smith and R. N. Martins (2016). “Plasma apolipoprotein 3 as a potential biomarker for Alzheimer's disease: Australian Imaging, Biomarkers and Lifestyle study of aging.” Alzheimers Dement (Amst) 3: 18-26.

Higgins, J. P., S. G. Thompson, J. J. Deeks and D. G. Altman (2003). “Measuring inconsistency in meta-analyses.” Bmi 327(7414): 557-560.

Kuleshov, M. V., M. R. Jones, A. D. Rouillard, N. F. Fernandez, Q. Duan, Z. Wang, S. Koplev, S. L. Jenkins, K. M. Jagodnik, A. Lachmann, M. G. McDermott, C. D. Monteiro, G. W. Gundersen and A. Ma'ayan (2016). “Enrichr: a comprehensive gene set enrichment analysis web server 2016 update.” Nucleic Acids Res 44(W1): W90-97.

Lusardi, T. A., J. I. Phillips, 3. T. Wiedrick, C. A. Harrington, B. Lind, J. A. Lapidus, 3. F. Quinn and J. A. Saugstad (2017). “MicroRNAs in Human Cerebrospinal Fluid as Biomarkers for Alzheimer's Disease.” J Alzheimers Dis 55(3): 1223-1233.

McKhann, G., D. Drachman, M. Folstein, R. Katzman, D. Price and E. M. Stadlan (1984). “Clinical diagnosis of Alzheimer's disease: report of the NINCDS-ADRDA Work Group under the auspices of Department of Health and Human Services Task Force on Alzheimer's Disease.” Neurology 34(7): 939-944.

O'Bryant, S. E., S. Lista, R. A. Rissman, M. Edwards, F. Zhang, J. Hall, H. Zetterberg, S. Lovestone, V. Gupta, N. Graff-Radford, R. Martins, A. Jeromin, S. Waring, E. Oh, M. Kling, L. D. Baker and H. Hampel (2016). “Comparing biological markers of Alzheimer's disease across blood fraction and platforms: Comparing apples to oranges.” Alzheimers Dement (Amst) 3: 27-34.

Ryan, M. M., D. Guévremont, B. G. Mockett, W. C. Abraham and J. M. Williams (2018). “Circulating Plasma microRNAs are Altered with Amyloidosis in a Mouse Model of Alzheimer's Disease.” J. Alzheimers Dis 66(2): 835-852.

Swarbrick, S., N. Wragg, S. Ghosh and A. Stolzing (2019). “Systematic Review of miRNA as Biomarkers in Alzheimer's Disease.” Mol Neurobiol 56(9): 6156-6167.

Takousis, P., A. Sadlon, J. Schulz, I. Wohlers, V. Dobricic, L. Middleton, C. M. Lill, R. Perneczky and L. Bertram (2019). “Differential expression of microRNAs in Alzheimer's disease brain, blood, and cerebrospinal fluid.” Alzheimers Dement 15(11): 1468-1477.

Wiedrick, J. T., J. I. Phillips, T. A. Lusardi, T. J. McFarland, B. Lind, U. S. Sandau, C. A. Harrington, J. A. Lapidus, D. R. Galasko, 3. F. Quinn and J. A. Saugstad (2019). “Validation of MicroRNA Biomarkers for Alzheimer's Disease in Human Cerebrospinal Fluid.” J Alzheimers Dis 67(3): 875-891.

Zhu, H. C., L. M. Wang, M. Wang, B. Song, S. Tan, J. F. Teng and D. X. Duan (2012). “MicroRNA-195 downregulates Alzheimer's disease amyloid-13 production by targeting BACE1.” Brain Res Bull 88(6): 596-601.

1.-38. (canceled)

39. A method for treating high amyloid-13 load in the brain of a human comprising:
(i) measuring the levels of miR-142-3p and miR-186-5p in a body fluid sample obtained from the human;
(ii) comparing the measured levels of miR-142-3p and miR-186-5p from (i) against a control sample from a human not having high amyloid-β load in the brain,
wherein, a measured level of miR-142-3p and miR-186-5p in the body fluid sample that is higher or lower relative to the control sample is predictive that the human has a high amyloid-13 load in the brain; and
(iii) treating the human who has a high amyloid-β load in the brain determined according to steps (i) and (ii) by administering an effective amount of a treatment selected from the group consisting of:
(a) an anti-amyloid antibody;
(b) a cholinesterase inhibitor;
(c) memantine; and
(d) any combination of (a) to (c)
to the human, thereby reducing the high amyloid-β load in the brain and improving cognitive function.

40. The method according to claim 39 which further comprises measuring the level of miR-122-5p.

41. The method according to claim 39 which further comprises measuring the level of miR-652-3p.

42. The method according to claim 40 which further comprises measuring the level of miR-652-3p.

43. The method according to any one of claims 39 to 42 which further comprises measuring the level of miR-335-5p.

44. The method according to any one of claims 39 to 42 which further comprises measuring the levels of miR-324-5p.

45. The method according to any one of claims 39 to 42 which further comprises measuring the level of miR-425-5p.

46. The method according to claim 43 which further comprises measuring the level of miR-425-5p.

47. The method according to claim 44 which further comprises measuring the level of miR-425-5p.

48. The method according to any one of claims 39 to 42 which further comprises measuring the level of miR-27a-3p.

49. The method according to claim 43 which further comprises measuring the level of miR-27a-3p.

50. The method according to claim 44 which further comprises measuring the level of miR-27a-3p.

51. The method according to claim 45 which further comprises measuring the level of miR-27a-3p.

52. The method according to claim 46 which further comprises measuring the level of miR-27a-3p.

53. The method according to claim 47 which further comprises measuring the level of miR-27a-3p.

54. The method according to any one of claims 39 to 42 which further comprises measuring the level of:
(i) miR-29c-3p;
(ii) miR-30c-5p;
(iii) miR-143-3p;
(iv) miR-199a-3p;
(v) miR-29c-3p and miR-30c-5p;
(vi) miR-29c-3p and miR-143-3p;
(vii) miR-29c-3p and miR-199a-3p;
(viii) miR-30c-5p and miR-143-3p;
(ix) miR-30c-5p and miR-199a-3p;
(x) miR-143-3p and miR-199a-3p;
(xi) miR-29c-3p, miR-30c-5p and miR-143-3p;
(xii) miR-29c-3p, miR-30c-5p and miR-199a-3p;
(xiii) miR-29c-3p, miR-143-3p and miR-199a-3p;
(xiv) miR-30c-5p, miR-143-3p and miR-199a-3p; and
(xv) miR-29c-3p, miR-30c-5p, miR-143-3p and miR-199a-3p.

55. The method according to claim 43 which further comprises measuring the level of:
(i) miR-29c-3p;
(ii) miR-30c-5p;
(iii) miR-143-3p;
(iv) miR-199a-3p;
(v) miR-29c-3p and miR-30c-5p;
(vi) miR-29c-3p and miR-143-3p;
(vii) miR-29c-3p and miR-199a-3p;
(viii) miR-30c-5p and miR-143-3p;
(ix) miR-30c-5p and miR-199a-3p;
(x) miR-143-3p and miR-199a-3p;
(xi) miR-29c-3p, miR-30c-5p and miR-143-3p;
(xii) miR-29c-3p, miR-30c-5p and miR-199a-3p;
(xiii) miR-29c-3p, miR-143-3p and miR-199a-3p;
(xiv) miR-30c-5p, miR-143-3p and miR-199a-3p; and

56. The method according to claim 44 which further comprises
(i) miR-29c-3p;
(ii) miR-30c-5p;
(iii) miR-143-3p;
(iv) miR-199a-3p;
(v) miR-29c-3p and miR-30c-5p;
(vi) miR-29c-3p and miR-143-3p;
(vii) miR-29c-3p and miR-199a-3p;
(viii) miR-30c-5p and miR-143-3p;
(ix) miR-30c-5p and miR-199a-3p;
(x) miR-143-3p and miR-199a-3p;
(xi) miR-29c-3p, miR-30c-5p and miR-143-3p;
(xii) miR-29c-3p, miR-30c-5p and miR-199a-3p;
(xiii) miR-29c-3p, miR-143-3p and miR-199a-3p;
(xiv) miR-30c-5p, miR-143-3p and miR-199a-3p; and
(xv) miR-29c-3p, miR-30c-5p, miR-143-3p and miR-199a-3p.

57. The method according to claim 46 which further comprises measuring the level of:
(i) miR-29c-3p;
(ii) miR-30c-5p;
(iii) miR-143-3p;
(iv) miR-199a-3p;
(v) miR-29c-3p and miR-30c-5p;
(vi) miR-29c-3p and miR-143-3p;
(vii) miR-29c-3p and miR-199a-3p;
(viii) miR-30c-5p and miR-143-3p;
(ix) miR-30c-5p and miR-199a-3p;
(x) miR-143-3p and miR-199a-3p;
(xi) miR-29c-3p, miR-30c-5p and miR-143-3p;
(xii) miR-29c-3p, miR-30c-5p and miR-199a-3p;
(xiii) miR-29c-3p, miR-143-3p and miR-199a-3p;
(xiv) miR-30c-5p, miR-143-3p and miR-199a-3p; and

58. The method according to claim 47 which further comprises measuring the level of:
(i) miR-29c-3p;
(ii) miR-30c-5p;
(iii) miR-143-3p;
(iv) miR-199a-3p;
(v) miR-29c-3p and miR-30c-5p;
(vi) miR-29c-3p and miR-143-3p;
(vii) miR-29c-3p and miR-199a-3p;
(viii) miR-30c-5p and miR-143-3p;
(ix) miR-30c-5p and miR-199a-3p;
(x) miR-143-3p and miR-199a-3p;
(xi) miR-29c-3p, miR-30c-5p and miR-143-3p;
(xii) miR-29c-3p, miR-30c-5p and miR-199a-3p;
(xiii) miR-29c-3p, miR-143-3p and miR-199a-3p;
(xiv) miR-30c-5p, miR-143-3p and miR-199a-3p; and
(xv) miR-29c-3p, miR-30c-5p, miR-143-3p and miR-199a-3p.

59. The method according to claim 52 which further comprises measuring the level of:
(i) miR-29c-3p;
(ii) miR-30c-5p;
(iii) miR-143-3p;
(iv) miR-199a-3p;
(v) miR-29c-3p and miR-30c-5p;
(vi) miR-29c-3p and miR-143-3p;
(vii) miR-29c-3p and miR-199a-3p;
(viii) miR-30c-5p and miR-143-3p;
(ix) miR-30c-5p and miR-199a-3p;
(x) miR-143-3p and miR-199a-3p;
(xi) miR-29c-3p, miR-30c-5p and miR-143-3p;
(xii) miR-29c-3p, miR-30c-5p and miR-199a-3p;
(xiii) miR-29c-3p, miR-143-3p and miR-199a-3p;
(xiv) miR-30c-5p, miR-143-3p and miR-199a-3p; and

60. The method according to claim 53 which further comprises measuring the level of:
(i) miR-29c-3p;
(ii) miR-30c-5p;
(iii) miR-143-3p;
(iv) miR-199a-3p;
(v) miR-29c-3p and miR-30c-5p;
(vi) miR-29c-3p and miR-143-3p;
(vii) miR-29c-3p and miR-199a-3p;
(viii) miR-30c-5p and miR-143-3p;
(ix) miR-30c-5p and miR-199a-3p;
(x) miR-143-3p and miR-199a-3p;
(xi) miR-29c-3p, miR-30c-5p and miR-143-3p;
(xii) miR-29c-3p, miR-30c-5p and miR-199a-3p;
(xiii) miR-29c-3p, miR-143-3p and miR-199a-3p;
(xiv) miR-30c-5p, miR-143-3p and miR-199a-3p; and
(xv) miR-29c-3p, miR-30c-5p, miR-143-3p and miR-199a-3p.

61. The method according to claim 46, wherein when the human has a high amyloid-β load in the brain:
(a) the level of miR-142-3p in the sample is higher than the level of miR-142-3p in the control sample;
(b) the level of miR-186-5p in the sample is higher than the level of miR-186-5p in the control sample;
(c) the level of miR-122-5p in the sample is lower than the level of miR-122-5p in the control sample;
(d) the level of miR-652-3p in the sample is higher than the level of miR-652-3p in the control sample;
(e) the level of miR-335-5p in the sample is higher than the level of miR-335-5p in the control sample;
(f) the level of miR-324-5p in the sample is higher than the level of miR-324-5p in the control sample;
(g) the level of miR-425-5p in the sample is higher than the level of miR-425-5p in the control sample;
(h) the level of miR-27a-3p in the sample is higher than the level of miR-27a-3p in the control sample;
(i) the level of miR-29c-3p in the sample is higher than the level of miR-29c-3p in the control sample;
(j) the level of miR-30c-5p in the sample is higher than the level of miR-30c-5p in the control sample;
(k) the level of miR-143-3p in the sample is higher than the level of miR-143-3p in the control sample; and/or (I) the level of miR-199a-3p in the sample is higher than the level of miR-199a-3p in the control sample.

62. The method according to claim 39, wherein the measuring comprises an amplification-based method or an array-based method.

63. The method according to claim 39, wherein the measuring is performed using a methodology selected from polymerase chain reaction (PCR), real-time polymerase chain reaction (RT-PCR), qualitative polymerase chain reaction (qPCR), rolling circle amplification, Nanostring analysis, northern blot analysis, branched DNA (bDNA) signal amplification, in situ hybridization, sequence-based methods, ribonuclease protection assay and mass spectroscopy.

64. The method according to claim 39, wherein the body fluid sample is selected from plasma, serum, white blood cells, whole blood and cerebral spinal fluid.

65. The method according to claim 39, wherein the body fluid sample is plasma.

66. The method according to claim 39, wherein the anti-amyloid antibody is aducanumab.
